(19) United States
(12) Patent Application Publication
(10) Pub. No.: US 2025/0057936 A1
(43) Pub. Date: Feb. 20, 2025

(54) VACCINE COMPOSITION

(71) Applicant: Lionel Scott, Douglas (IM)

(72) Inventor: Lionel Scott, Douglas (IM)

(21) Appl. No.: 18/910,182

(22) Filed: Oct. 9, 2024

(30) Foreign Application Priority Data
Apr. 9, 2022 (GB) 2205231.0
Mar. 29, 2023 (WO) PCT/GB2023/000019

(51) Int. Cl. A61K 39/145; A61K 9/00; A61K 9/16; A61K 39/00; A61K 39/215; A61P 31/16; C12N 7/00

(52) U.S. Cl. CPC A61K 39/145; A61K 9/0043; A61K 9/1652; A61K 9/1682; A61K 39/215; A61P 31/16; C12N 7/00; A61K 2039/543; A61K 2039/55505; A61K 2039/55566; A61K 2039/575; A61K 2039/70; C12N 2760/16134; C12N 2760/20034

(57) ABSTRACT

Respiratory virus vaccine compositions for nasal administration to a mammal comprising a hygroscopic gel-forming material, at least one isolated bioactive respiratory virus immunogen, and an adjuvant, kits, receptacles, uses therefor, and methods of manufacture thereof.

The present invention relates to vaccine compositions against respiratory viruses, methods of manufacture, kits and uses therefor. In particular, the invention relates to vaccine compositions against respiratory viruses for nasal delivery comprising hydroxypropyl methylcellulose (HPMC), an immunogenic protein or polypeptide and an adjuvant, methods of manufacture, kits and uses therefor.

Respiratory viruses from diverse viral groups are responsible for causing disease and death in human beings. Examples of respiratory virus types responsible for such effects include the coronaviruses, influenza viruses, such as strains of influenza A and B, parainfluenza viruses, metapneumoviruses, respiratory syncytial viruses, rhinoviruses, bocaviruses and the like.

Taking but two examples of respiratory disease caused by viruses, such as influenza and COVID-19, mortality rates in human beings are concerning. Influenza virus strains have been shown in one study to cause an estimated average of 389,000 respiratory-related deaths worldwide per annum over the period 2002-2011 (Paget J. et al. 2019 Journal of Global Health December 2019, Vol. 9 No. 2 020421, 1-12. DOI: 10.7189/jogh.09.020421) whereas in another study (Iuliano A. D. et al. 2018 Lancet; 391:1285-300, Medline: 29248255 doi: 10.1016/S0140-6736 (17) 332293-2) the results of which have recently been adopted by the World Health Organisation, the number of people dying from respiratory diseases linked to seasonal flu worldwide each year is put at up to 650,000.

The current pandemic of COVID-19 disease due to variants of the causative agent, the SARS-COV-2 virus, has resulted in several millions of deaths (approximately 6.16 million) and several hundreds of millions of confirmed cases of COVID-19 worldwide over two and a half years (approximately 489 million) as of 1 Apr. 2022 (Worldometer Info. https://www.worldometers.info).

While vaccination, the best current means available to save lives in countries having access thereto is on-going, countries having limited access to vaccines and containing very large populations of the total human population worldwide, numbering in the billions, remain at risk of contracting the disease and of possibly dying from it. The current vaccination programs take time, considerable effort, and huge cost to deploy to very large numbers of people. As a result, many hundreds of millions of people continue to miss out on vaccinations as demand rapidly exhausts current supply.

Many vaccines against COVID-19 disease and other respiratory viruses have been developed or are in development. Over ninety vaccines against COVID-19 disease are in clinical trials (Seifalian A. M. et al. COVID-19 Vaccines in Clinical Trials and their Mode of Action for Immunity Against the Virus. Current Pharmaceutical Design 2021, 27, 1-11; Muhammed Y. et al. SARS-COV-2 spike protein and RNA dependent RNA polymerase as targets for drug and vaccine development: A review. Biosafety and Health 3 2021 249-263 [https://doi.org/10.1016/j.bsheal.2021.07.003]; Lund F. E and Randall T. D. Scent of a Vaccine 2021 Science 373 Issue 6553 397-399 [DOI 10.1126/science.abg9857]). Particular emphasis is on the provision of mRNA vaccines (Pfizer-BioNtech [LNP-encapsulated trimer RNA] and Moderna [LNP-encapsulated RNA]), replication-defective recombinant adenoviral vector vaccines (Janssen-Johnson and Johnson, Astra Zeneca [non-replicating viral vector], Sputnik-V, and CanSino [non-replicating viral vector]), and inactivated vaccines (Sinopharm, Bharat Biotech and Sinovac). In addition, at least one protein vaccine against COVID-19 disease is in clinical trials [Novavax: full length recombinant SARS-COV-2 glycoprotein NP vaccine adjuvanted with Matrix M].

Many of the SARS-COV-2 vaccines (more than 90%) currently known to be in clinical trials are delivered by injection, such as by parenteral, intramuscular, or subcutaneous administration. Eight vaccines that are in clinical trials or have finished clinical trialling and are designed to be delivered intranasally include ChAdOx1-S (University of Oxford: uses a Chimp adenovirus vector [spike]); AdCOVID (Altimmune: uses an adenovirus 5 vector [RBD]; BBV154 (Bharat Biotech: uses a Simian adenovirus vector [spike]); DeINS1-nCOV-RBD LAIV (University of Hong Kong: uses a live attenuated influenza virus carrying SARS-COV-2 spike-RBD [RBD]); MV-014-212 (Meisse Vaccines: uses a live attenuated respiratory syncytial virus carrying a SARS-COV-2 immunogen [spike]); COVI-VAC (Codagenix: uses a live attenuated SARS-COV-2; and CIBG-669 (Center for Genetic Engineering and Biotechnology, Cuba: uses RBD linked to the hepatitis B virus core antigen, a potent stimulator of T cells); and the Sputnik V (Gamaleya Institute, Russia) nasal vaccine which is a heterologous COVID-19 vaccine containing two components, a recombinant adenovirus type 26 (rAd26) vector and a recombinant adenovirus type 5 (rAd5) vector which both carry the SARS-COV-2 spike glycoprotein. The vaccine is offered in both a frozen (Gam-COVID-Vac) and freeze-dried formulation (lyophilizate; Gam-COVID-Vac Lyo). The same two adenovirus non-replicating viral vectors known from the intramuscularly administered sputnik V vaccine are delivered as a nasal liquid spray in two doses. The vaccine components are the same ones as used in the two component form given intramuscularly except that “ . . . instead of a needle, a nozzle is put on” and “we are just administering the same vaccine as a nasal spray” “(TASS news agency report) according to Alexander Gintsburg, the Director of the Gamaleya National Research Center (sic) for Epidemiology and Microbiology, as relayed by Reuters, Jun. 12, 2021. However, all of the above-mentioned nasal vaccines are applied intranasally in liquid form or as a liquid spray. None of the prior art nasal vaccine formulations and/or components thereof employs components of the present invention, such as HPMC, and any other components, in powdered form.

mRNA vaccines have certain advantages over others in that they are flexible and efficient in immunogen design and relatively easy to manufacture. The BNT162b2 vaccine is one example of a COVID-19 mRNA vaccine which has been evaluated in successful clinical trials (Mulligan, M. J et al. Phase I/II study of COVID-19 RNA vaccine BNT162b1 in adults. Nature 2020, 586, 589-593; Polack, F. P et al. Safety and Efficacy of the BNT162b2mRNA COVID-19 Vaccine. N. Engl. J. Med. 2020, 383, 2603-2615.) and administered in certain national COVID-19 vaccination campaigns worldwide (Harris, R. J et al. Effect of Vaccination on Household Transmission of SARS-COV-2 in England. N. Engl. J. Med. 2021, 385, 759-760; Butt, A. A et al. SARS-CoV-2 Vaccine Effectiveness in a High-Risk National Population in a Real-World Setting. Ann. Intern. Med. 2021, 174, 1404-1408; Dagan, N. et al. BNT162b2 mRNA Covid-19 Vaccine in a Nationwide Mass Vaccination Setting. N. Engl. J. Med. 2021, 384, 1412-1423; Rossman, H. et al. COVID-19 dynamics after a national immunization program in Israel. Nat. Med. 2021, 27, 1055-1061).

BNT162b2 itself is a lipid nanoparticle (LNP)-encapsulated, nucleoside-modified RNA vaccine (modRNA) encoding the full length SARS-COV-2 spike(S) protein, modified by two proline mutations to ensure antigenically optimal pre-fusion conformation mimics the intact virus to elicit virus-neutralizing antibodies (Walsh, E. E. et al. Safety and Immunogenicity of Two RNA-Based COVID-19 Vaccine Candidates. N. Engl. J. Med. 2020, 383, 2439-2450).

However, an issue with BNT162b2 at this time is that the safety profile is currently available only for short-term clinical studies. Furthermore, certain adverse side effects of BNT162b2 have been reported in the scientific literature. Such side effects include pericarditis, arrhythmia, deep-vein thrombosis, pulmonary embolism, myocardial infarction, intracranial haemorrhage, and thrombocytopenia (Polack, F. P. supra; Fan, B. E. et al. Cerebral venous thrombosis post BNT162b2 mRNA SARS-COV-2 vaccination: A black swan event. Am. J. Hematol. 2021, 96, E357-E361; Larson, K. F. et al. Myocarditis After BNT162b2 and mRNA-1273 Vaccination. Circulation 2021, 144, 506-508; Menni, C. et al. Vaccine side-effects and SARS-COV-2 infection after vaccination in users of the COVID Symptom Study app in the UK: A prospective observational study. Lancet Infect. Dis. 2021, 21, 939-949; Hansen, T. et al. First case of post mortem study in a patient vaccinated against SARS-COV-2. Int. J. Infect. Dis. 2021, 107, 172-175; Kadali, R. A. K. et al. Side effects of BNT162b2 mRNA COVID-19 vaccine: A randomized, cross-sectional study with detailed self-reported symptoms from healthcare workers. Int. J. Infect. Dis. 2021, 106, 376-381; Parkash, O. et al. Acute Pancreatitis: A Possible Side Effect of COVID-19 Vaccine. Cureus 2021, 13, e14741; Mazzatenta, C. et al. Purpuric lesions on the eyelids developed after BNT162b2 mRNA COVID-19 vaccine: Another piece of SARS-COV-2 skin puzzle? J. Eur. Acad. Dermatol. Venereol. 2021, 35, e543-e545; Lee, E. J. et al. Thrombocytopenia following Pfizer and Moderna SARS-COV-2 vaccination. Am. J. Hematol. 2021, 96, 534-537; Ishay, Y. et al. Autoimmune phenomena following SARS-COV-2 vaccination. Int. Immunopharmacol. 2021, 99, 107970; Das, B. B. et al. Myopericarditis following mRNA COVID-19 Vaccination in Adolescents 12 through 18 Years of Age. J. Pediatr. 2021, 238, 26-32.e1; McLaurin-Jiang, S. et al. Maternal and Child Symptoms Following COVID-19 Vaccination Among Breastfeeding Mothers. Breastfeed. Med. 2021, 16, 702-709; Barda, N. et al. Safety of the BNT162b2 mRNA Covid-19 Vaccine in a Nationwide Setting. N. Engl. J. Med. 2021, 385, 1078-1090). Transient hepatic effects are inducible by LNP delivery systems (Tanaka, H. et al. Delivery of Oligonucleotides Using a Self-Degradable Lipid-Like Material. Pharmaceutics 2021, 13, 544; Sedic, M. et al. Safety Evaluation of Lipid Nanoparticle-Formulated Modified mRNA in the Sprague-Dawley Rat and Cynomolgus Monkey. Vet. Pathol. 2018, 55, 341-354; Sato, Y. et al Highly specific delivery of siRNA to hepatocytes circumvents endothelial cell-mediated lipid nanoparticle-associated toxicity leading to the safe and efficacious decrease in the hepatitis B virus. J. Control. Release 2017, 266, 216-225; Heidel, J. D. et al. Administration in non-human primates of escalating intravenous doses of targeted nanoparticles containing ribonucleotide reductase subunit M2 siRNA. Proc. Natl. Acad. Sci. USA 2007, 104, 5715-5721). Nevertheless, it has also been shown that the empty LNP (i.e. without modRNA included) alone does not introduce any significant liver injury (Tanaka H. et al. supra).

Other studies also report adverse side effects with other types of vaccines (Baden, L. R. et al. Efficacy and Safety of the mRNA-1273 SARS-COV-2 Vaccine. N. Engl. J. Med. 2021, 384, 403-416; Sadoff, J. et al. Safety and Efficacy of Single-Dose Ad26.COV2.S Vaccine against Covid-19. N. Engl. J. Med. 2021, 384, 2187-2201; Eichinger, S. et al. Thrombotic Thrombocytopenia after ChAdOx1 nCOV-19 Vaccination. Reply. N. Engl. J. Med. 2021, 385, e11; Doroftei, B. et al. Mini-Review Discussing the Reliability and Efficiency of COVID-19 Vaccines. Diagnostics 2021, 11, 579).

A recent study has also shown that SARS-COV-2 RNAs can be reverse-transcribed and integrated into the genome of cultured human cells (Zhang, L. et al. Reverse-transcribed SARS-COV-2 RNA can integrate into the genome of cultured human cells and can be expressed in patient-derived tissues. Proc. Natl. Acad. Sci. USA 2021, 118, e2105968118).

Alden M. et al. (‘Intracellular Reverse Transcription of Pfizer BioNTech COVID-19 mRNA Vaccine BNT162b2 In Vitro in Human Liver Cell Line’ Curr. Issues Mol. Biol. 2022, 44, 1115-1126. https://doi.org/10.3390/cimb44030073) reported that reverse transcription of the Pfizer BioNTech Covid-19 mRNA Vaccine BNT162b2 occurred in liver cell lines in vitro coupled with changes of gene expression and distribution of the long interspersed nuclear element-1 (LINE-1), an endogenous reverse transcriptase. Other vaccines are in development that make use of further exotic components. One example is of a plant-derived virus-like particle vaccine against COVID-19 (Ward B. J. et al. Phase 1 randomized trial of a plant-derived virus-like particle vaccine for COVID-19. Nature Medicine 2021 https://doi.org/10.1038/s41591-021-01370-1). The vaccine disclosed by Ward et al. is described as injectable, and the virus-like particle within the vaccine composition being of a similar size and shape (80-120 nM) to that of the SARS-COV-2 virus. The vaccine composition needs to be stored between 2-8° C. before use. There is no suggestion that components, such as hydroxypropyl methyl cellulose (HPMC) particles could be used as a carrier for an immunogenic component in a nasally-applied vaccine.

Still further vaccines in development for other viruses make use of cellulose-based materials such as cellulose-based polyelectrolyte complexes for DNA vaccine delivery (Song Y et al. Cellulose-based polyelectrolyte complex nanoparticles for DNA vaccine delivery. 2014 Biomater. Sci. 2, 1440-1449 DOI: 10.1039/c4bm00202d). The vaccine was developed against infection with bovine viral diarrhoea (BDVD) and employed inter alia carboxymethyl cellulose (CMC) and positively charged quaternized cellulose mixed together in an aqueous medium forming CMC-QC nanoparticles that may be used as a carrier for DNA vaccines. There is no suggestion that components such as hydroxypropyl methyl cellulose (HPMC) particles could be used as a carrier for an immunogenic component in a nasally-applied vaccine.

Microspheres made up of a blend of chitosan and HPMC are described as an apparent adjuvant for delivering tetanus toxoid vaccine stabilised with heparin and administered in a liquid format (Arthanari S et al. Chitosan-HPMC-blended microspheres as a vaccine carrier for the delivery of tetanus toxoid 2016 Artificial Cells, Nanomedicine, and Biotechnology, 44:2, 517-523, DOI: 10.3109/21691401.2014.966193). There is no suggestion that components such as hydroxypropyl methyl cellulose (HPMC) particles per se could be used as a carrier for an immunogenic component in a nasally-applied vaccine.

Microcrystalline cellulose for delivery of recombinant protein-based antigen against Erysipelas have been proposed and demonstrated in mice (Jeon W. et al. 2018 ‘Microcrystalline Cellulose for Delivery of Recombinant Protein-Based Antigen against Erysipelas in Mice’ Biomed Research International Volume 2018, Article ID 7670505 https://doi.org/10.1155/2018/7670505) The vaccine produced included antigen made up of recombinant CBD-SpaA with an incorporated S₃N₁₀ linker. Antigen-immobilised Avicel (microcrystalline) vaccine was subcutaneously injected into mice. However, there is no suggestion that components such as hydroxypropyl methyl cellulose (HPMC) particles could be used as a carrier for an immunogenic component in a nasally-applied vaccine.

The prior art clearly shows that there are potential if not actual inherent risks in taking mRNA and/or DNA vaccines and that dangerous side effects have been observed in vulnerable persons vaccinated with mRNA and/or DNA vector vaccines. There is a clear need to develop vaccines that are safer and simpler to apply and which carry a far lower risk of adverse reactions arising, post-vaccination.

Those skilled in the art know that nasal infections and vaccinations elicit an immunoglobulin A (IgA) response in both serum and respiratory fluids, whereas intramuscular vaccines primarily elicit IgG. IgA is important in the nasal passages where it is actively transported across the epithelium and released into the airway lumen as a dimer bound to a secretory component that allows it to neutralise viruses, such as SARS-COV-2, more effectively (Wang Z. et al. 2021 Sci. Transl. Med. 13, No. 577 [DOI: 10.1126/scitranslmed.abf1555]). By contrast, IgG enters and protects the lower lung through passive transudation across the thin alveolar epithelium (Renegar K. B. et al. 2004 J. Immunol. 173, 1978). Furthermore, compared to intramuscular vaccines, intranasal vaccines can provide two additional layers of protection: vaccine-elicited IgA and resident memory B and T cells in the respiratory mucosa provide an effective barrier to infection at those sites, and even if infection does occur, resident memory B and T cells which encounter antigen earlier and respond more quickly than systemic memory cells impede viral replication and reduce viral shedding and transmission (Lund F. E. and Randall. T. D. 2021 Science (10.1126/science.abg9857) Scent of a Vaccine). It is known from pre-clinical studies of adenovirus-vector vaccines expressing the SARS-COV-2 spike host receptor protein or its receptor binding domain (RBD) demonstrate that intranasal delivery triggers long-lasting, virus-neutralising serum IgG responses as well as antigen-specific IgA and CD8+ T cells in the respiratory tract (Hassan A. O. 2021 bioRxiv 10.1101/2021.05.08.443267; King R. G. et al. 2020 bioRxiv 10.1101/2020.10.10.331348; and van Doremalen N. et al. 2021 bioRxiv 10.1101/2021.01.09.426058). However, none of these cited references provide any allusion specific or implied to a composition comprising HPMC as a carrier and gelling agent for use in a vaccine formulation suitable for nasal administration.

Yang, J. et al. in ‘A vaccine targeting the RBD of the S protein of SARS-COV-2 induces protective immunity’, [Nature (2020), Vol. 586 (https://doi.org/10.1038/s41586-020-2599-8] reported that during the most critical step during infection of human beings, SARS-COV-2 uses its Spike protein receptor-binding domain (S-RBD) to engage with the host cell receptor angiotensin-converting enzyme 2 (ACE2) and show that a recombinant vaccine comprising residues 319-545 of the S-RBD could induce a potent functional antibody response in immunized mice, rabbits, and non-human primates. The sera from the immunized animals blocked RBD binding to ACE2 expressed on the cell surface and neutralized the infection by SARS-COV-2 pseudovirus and live SARS-COV-2 in vitro. Importantly, the vaccination also provided protection in non-human primates from SARS-COV-2 challenge in vivo. Elevated levels of RBD-specific antibodies were also found in the sera from patients with COVID-19. This study highlighted the importance of the RBD domain in the SARS-COV-2 vaccine design and provided the rationale for the development of a protective vaccine through the induction of antibody against the RBD domain. However, Yang et al provide no allusion specific or implied to the provision of a composition comprising HPMC as a carrier and gelling agent for use in a vaccine formulation suitable for nasal administration.

Markus Hoffman et al. (Cell, Volume 181, Issue 2, P 271-280.E8, Apr. 16, 2020 ‘SARS-CoV-2 Cell Entry Depends on ACE2 and TMPRSS2 and Is Blocked by a Clinically Proven Protease Inhibitor’ (Open Access published Mar. 5, 2020 DOI: http://doi.org/10.1016/j.cell.2020.02.052) reported that convalescent SARS patients exhibit a neutralizing antibody response directed against viral S protein and investigated whether such antibodies blocked SARS-2-S-driven entry. Furthermore, Hoffman et al investigated whether a transmembrane protease serine 2 (TMPRSS2), a primer for SARS-COV-2 S protein (spike protein) usage was required for SARS-COV-2 infection of lung cells and appeared to conclude that it was.

However, Hoffman et al provide no allusion specific or implied to the provision of a composition comprising HPMC as a carrier and gelling agent for use in a vaccine formulation suitable for nasal administration.

Thus, the prior art teaches of promising immunogenic components for use as biologically active agents against SARS-COV-2 related disease, such as SARS-COV-2 spike protein and immunogenic parts thereof, SARS-COV-2 nucleocapsid protein and immunogenic parts thereof, spike protein receptor binding domain (S-RBD) and immunogenic parts thereof, the S1 subunit of the spike(S) protein and immunogenic components thereof such as the C-domain, and the transmembrane protease TMPRSS2 and immunogenic components thereof. However, it is also clear that the teaching of the prior art does not allude to the presentation of immunogenic components formulated in any form, such as in a powdered form, or in a liquid form that utilise HPMC as a carrier and gelling agent for nasal administration to the nasal mucosa. Thus, vaccine composition formulations of the present invention represent a novel approach for the administration of vaccines to patients and are considered to obviate many of the problems associated with conventional vaccine formulations that may be administered nasally, orally, parenterally, subcutaneously, intramuscularly, or the like.

There is a clear need for respiratory virus vaccine compositions which are simpler to administer and that do not require the use of syringes fitted with needles or other skin puncturing equipment.

There is a need for vaccine compositions which include components that do not have an absolute requirement for cold transport and/or cold storage conditions.

There is a need for vaccine compositions to be safer in terms of their side effects in all vaccinated persons and especially in medically vulnerable persons.

There is a need for vaccine compositions that are capable of driving a powerful immune response both in terms of generating significant levels of IgA and IgG antibodies.

There is a need for nasal vaccine compositions that form gels to prevent the ingress of viruses into, and shedding of viruses from, the nasal cavity of vaccinated individuals. Concomitantly, the formed gel slows down and/or substantially prevents the egress of active vaccine components from the nasal cavity.

Thus, it is an object of the present invention to substantially reduce the need for employing cold temperature transport and/or storage conditions in the transportation and/or storage of vaccine compositions.

It is another object of the present invention to provide vaccine compositions that are simple to administer and which do not require the use of specialised applicators such as syringes fitted with needles or other skin puncturing equipment or the use of other specialised equipment for administering vaccines to patients.

It is a further object of the present invention to provide vaccine compositions that are substantially safer to use than mRNA and/or DNA-based vaccine compositions.

It is yet a further object of the invention to provide vaccine compositions that prevent the ingress of viruses into, and the shedding of viruses from the nasal cavity of individuals vaccinated with compositions of the invention.

These and other objects will become apparent from the following description.

According to the present invention there is provided a respiratory virus vaccine composition for nasal administration to a mammal comprising an hygroscopic gel-forming material, at least one isolated bioactive respiratory virus immunogen, and an adjuvant.

Respiratory virus vaccine compositions of the invention typically take the form of a dry particulate powder wherein the particles have a mean particle size diameter of ≥10 μm to ≤400 μm. Preferably, the mean particle size diameter lies within the range≥50 μm to ≤300 μm, more preferably within the range≥50 μm to ≤150 μm, such as in the range≥60 μm to ≤140 μm, or ≥80 μm to ≤120 μm.

The respiratory virus vaccine compositions of the invention include an immunogen component, the said immunogen being made up of one or more isolated immunogenic viral proteins or immunogenic viral carbohydrates either in liquid or in powder form. Preferably, the immunogen component comprises one or more isolated viral proteins in powder form when applied to the nasal cavity.

The hygroscopic gel-forming material of an inventive composition of the invention is one that must be capable of forming a gel layer or barrier on contact with moisture on the nasal mucosa within the nasal cavity and may be selected from any suitable hygroscopic material, such as natural polymers of starch, collagen, lecithin (in the form of lecithin stabilised polymeric micelles, carrageenan and hydroxypropylmethyl cellulose. Preferably, the hygroscopic gel-forming material is hydroxypropylmethyl cellulose.

Where the hygroscopic gel-forming material is hydroxypropylmethyl cellulose, the mean viscosity of the particles of HPMC per se, prior to mixing with the other components of the respiratory virus vaccine composition lies within 10 to 20 Pa·S, at 20° C. in a 2% aqueous solution, such as, 10 Pa·S to 17 Pa·S at 20° C. in a 2% aqueous solution or 12 Pa·S to 15 Pa·S at 20° C. in a 2% aqueous solution.

Thus, in a preferment of the present invention, there is provided a respiratory virus vaccine composition in the form of a dry particulate powder comprising:
i) dry powder hydroxypropyl methylcellulose (HPMC) particles; and
ii) at least one isolated bioactive respiratory virus immunogen; and
iii) an adjuvant,
   wherein the mean viscosity of the dry powder HPMC particles per se lies within 10 to 20 Pa·S, at 20° C. in a 2% aqueous solution, such as, 10 Pa·S to 17 Pa·S at 20° C. in a 2% aqueous solution.

The mean particle size diameter of the HPMC particles per se may be of any size within the range≥10 μm to ≤400 μm, such as ≥50 μm to ≤300 μm, ≥50 μm to ≤150 μm, ≥60 μm to ≤140 μm or ≥80 μm to ≤120 μm. Preferably, the mean particle size diameter of particles of HPMC particles of use in the invention is ≥80 μm to ≤120 μm.

The skilled addressee will appreciate that the viscosity of the entire hydroxypropyl-methylcellulose containing dry particulate powder respiratory virus vaccine composition of the invention will have a higher viscosity than the HPMC particles per se, such as from 22 Pa·s+/−2 Pa·S to 40 Pa·s.+/−5 Pa·S at 20° C. in a 3.6% aqueous solution.

Typically, the mean viscosity of the entire dry particulate powder respiratory virus vaccine composition of the invention lies within the range 26+/−2 Pa·S to 34+/−0.5 Pa·S. at 20° C. in a 3.6% aqueous solution. In a further refinement the mean viscosity may be 28 Pa·S+/−2 to 32 Pa·S+/−5 Pa·S. at 20° C. in a 3.6% aqueous solution. The skilled addressee will appreciate that the viscosity of compositions of the invention may be subtly different from one day to the next depending on prevailing ambient humidity and temperature whilst being made up.

Thus, in an additional aspect of the present invention, there is provided a respiratory virus vaccine composition in the form of a dry particulate powder consisting of:
i) dry powder particles of an hygroscopic gel-forming material; and
ii) at least one isolated bioactive respiratory virus immunogen; and
iii) an adjuvant,
   wherein the made up respiratory virus vaccine composition has a mean viscosity within the range 22 Pa·s+/−2 Pa·S to 40 Pa·s.+/−5 Pa·S at 20° C. in a 3.6% aqueous solution.

The ‘dry particulate powder’ is typically made up of particles of an hygroscopic gel-forming material such as carrageenan and/or HPMC particles, and at least one isolated bioactive respiratory virus immunogen. Where the gel forming material is made up of HPMC particles they can be of any size provided always that when admixed with other components, such as at least one isolated bioactive respiratory virus immunogen and adjuvant, the resultant mixture forms a gel layer on contact with moisture on the nasal mucosa. Typically, admixed compositions of the invention possesses a mean viscosity within the range 22 Pa·s+/−2 Pa·S to 40 Pa·s.+/−5 Pa·S at 20° C. in a 3.6% aqueous solution. The viscosities of compositions of the invention are measured using standard procedures as taught in the European Pharmacopeia Chapter 2.2.10 on Rheology Analysis and may be performed on a TA Discovery Hybrid Rheometer 1 (TA DHR1) from TA Instruments Inc., Wilmington, USA), or comparable Rheometer, and the mean viscosity of several samples calculated therefrom. A suitable mean particle size for HPMC particles of use in the invention has been found to be from 60 μm to 140 μm, although it is to be understood that the mean particle size of HPMC particles may also lie outside of this range, for example, between about 50 μm and about 150 μm as long as the respiratory virus vaccine composition of the invention has a mean viscosity as defined herein.

Typically, the mean viscosity of the entire dry particulate powder respiratory virus vaccine composition of the invention lies within the range 26+/−2 Pa·S to 34+/−0.5 Pa·S. In a further refinement the mean viscosity may be 32 Pa·S+/−5 Pa·S. Naturally, the skilled addressee will appreciate that dry particulate powders of the invention are particulate enough to form substantially uniform plumes in shape when discharged from a vaccine dispenser receptacle, such as from those described herein. Furthermore, the skilled addressee will appreciate that the viscosity of compositions of the invention may be subtly different from one day to the next depending on the ambient humidity conditions whilst such compositions are being made up.

Powdered gel forming material of use in the invention typically comprises the bulk of vaccine formulations of the instant invention. As one illustration, where the gel-forming material is HPMC, it comprises at least 90%, and preferably at least 91, 92, 93, 94, 95, 96, 97, 98, or 99% by total weight of compositions of the invention. Preferably, compositions of the invention containing an immunogen as defined herein typically comprise powdered HPMC making up at least 95, 96, 97, 98 or 99%, preferably 95% to 99% of the total weight of the composition depending on requirement.

In a preferred embodiment of the present invention, the ratio of immunogen to powdered HPMC in the composition (by weight) is between 0.1:9.9 and 1.9:8.1, 0.2:9.8 and 1.8:8.2, 0.5:9.5 and 1.5:8.5, 0.6:9.4 and 1.4:8.6, 0.8:9.2 and 1.2:8.8, 1.0:9.0 and 1.5:8.5, 1.5:8.5 and 2.0:8.0, 2.0:8.0 and 2.5:7.5, 2.5:7.5 and 3.0:7.0, 3.0:7.0 and 3.6:6.4, 3.6:6.4 and 4:6.0 or 2.5:7.5 and 3.6:6.4 depending on design.

In a preferred embodiment of the present invention, the immunogen makes up from 0.10% to ≤8%, preferably from 0.50% to 5%, or about 1%, 2% 3% or 4% or any value therein between of the total weight of the composition. It is to be understood that the amount of any individual isolated immunogen or isolated immunogens that may be utilised in the present invention will be dependent on the virus species, and/or the number of virus strains or species against which compositions of the invention are employed and will be measured in micrograms in the range of from ≥2 μg to ≤500 μg per dose, such as ≥20 μg to ≤150 μg, more preferably from ≥35 μg to ≤100 μg, such as ≥50 μg to ≤100 μg, ≥60 μg to ≤80 μg per dose or any amount therein between depending on virus species or strains. The above ratios are merely illustrative of the kinds of ratios of immunogen to HPMC that may be found in compositions of the invention and will vary between vaccine compositions of differing volume that are designed for specific purposes. Such ratios are typically designed to utilise amounts of immunogen as defined herein for any one dose.

A respiratory virus vaccine composition of the invention contains at least one isolated bioactive respiratory virus immunogen that is capable of eliciting an immune response in a host subject against one or more respiratory viruses selected from the group: coronaviruses, influenza viruses, such as strains of influenza A, parainfluenza viruses, metapneumoviruses, respiratory syncytial viruses, rhinoviruses, and bocaviruses.

Suitable candidate respiratory virus immunogens include corona virus immunogens such as from the SARS-COV-2 virus, wherein the immunogenic component is selected from: the SARS-COV-2 spike protein and immunogenic parts thereof, the trimeric spike protein and immunogenic parts thereof, the SARS-COV-2 nucleocapsid protein and immunogenic parts thereof, spike protein receptor binding domain (S-RBD) and immunogenic parts thereof, the S1 subunit of the spike(S) protein and immunogenic parts thereof such as the C-domain, and the transmembrane protease TMPRSS2 and immunogenic components thereof. Preferably, the immunogenic component against SARS-COV-2 is selected from a spike RBD protein and immunogenic components thereof. Other respiratory virus immunogenic components that may be employed in compositions of the invention include those listed herein, such as from influenza virus as described in publications such as Skalupka A. L. et al. 2021 (Universal Influenza Virus Neuraminidase Vaccine Elicits Protective Immune Responses against Human Seasonal and Pre-pandemic Strains J Virol. 2021 Aug. 10; 95 (17): e0075921. doi: 10.1128/JVI.00759-21. Epub 2021 Aug. 10) describes the use of a computationally optimized broadly reactive antigen (COBRA) neuraminidase (NA) surface protein for a vaccine design. A methodology was used to generate the N1-INA vaccine antigen that was designed to cross-react with avian, swine, and human influenza viruses of the N1 NA subtype. The elicited antibodies bound to NA proteins derived from A/California/07/2009 (H1N1)pdm09, A/Brisbane/59/2007 (H1N1), A/Swine/North Carolina/154074/2015 (H1N1), and A/Viet Nam/1203/2004 (H5N1) influenza viruses, with NA-neutralizing activity against a broad panel of HXN1 influenza strains. The N1-I COBRA NA antigen has the potential to be a complementary component in a multi-antigen universal influenza virus vaccine formulation that also contains haemagglutinin antigens (HA) antigens. HA consists of two distinct domains, the variable HA head domain, composed of part of HA1, and a stalk structure, composed of portions of HA1 and all of HA2. Traditional IAV vaccines depend on the generation of neutralizing antibodies (Abs) specific for the variable HA head domain, and thus the vaccines are strain specific. Less variable HA regions, including the stalk domain or/and conserved epitopes within the head domain are also targeted using different approaches including the use of headless HA, chimeric HA, mosaic HA, computationally-optimized broadly reactive antigens (COBRA), and “breathing” HA to make broad spectrum IAV vaccines; the respiratory syncytial virus (RSV) proteins from RSV A and RSV B, such as the G and F glycoprotein (such as PFP-1 and/or PFP-2 adsorbed onto alum hydroxide and PFP-3 adsorbed onto alum phosphate; and RSV A and RSV B co-purified F, G and matrix (M) proteins with either alum or other adjuvant such as polydicarboxylatophenoxy phosphazene; and BBG2Na, a peptide from the G glycoprotein conjugated to the albumin-binding domain of streptococcal protein G in alum. BBG2Na is a prokaryotically expressed fusion protein that consists of the central conserved region of the G glycoprotein from the RSV A Long strain (residues 130-230) fused to the albumin-binding domain of streptococcal protein G, which acts as a carrier protein; the parainfluenza virus (PIV) proteins F and N proteins of human PIV-3; the human metapneumovirus (HMPV) proteins such as protein F; rhinovirus (RV) proteins such as VP1, and VP3 from RV14 and conserved regions of VP4. Edylmayr J. et al. 2011 (Antibodies induced with recombinant VP1 from human rhinovirus exhibit cross-neutralisation Eur Respir J. 37 (1): 44-52. doi: 10.1183/09031936.00149109) produced recombinant VP1 of two distinct RV serotypes and demonstrated the production of neutralizing antibodies (Abs) for additional RV serotypes. In the most novel RV vaccine approach attempted to date, Glanville N. et al. 2013 (Cross-serotype immunity induced by immunization with a conserved rhinovirus capsid protein. PLOS Pathog. 9 (9): e1003669. doi: 10.1371/journal.ppat.1003669) identified a conserved region of the RV polyprotein encompassing VP4 and VP2 (known as VP0), and generated RV16 VP0-VP0 which was immunogenic when combined with a strong adjuvant, again, suggesting that native capsid configurations are needed to induce neutralizing Abs. The most recent study investigating VP0 immunization has determined the immunodominant epitope for Abs corresponds to the previously identified Nlm-II region of VP2 (Sam N. J. et al. 2019 ‘Epitope mapping of antibodies induced with a conserved rhinovirus protein generating protective anti-rhinovirus immunity. Vaccine.’ 2019; 37 (21): 2805-13. doi: 10.1016/j.vaccine.2019.04.018); and the human bocavirus (HBoV) recombinant protein HBoV viral capsid protein 2-virus like particles (HBoV VP2-VLP), Deng Z-H et al. 2014 (Immunogenicity of recombinant human bocavirus-1,2 VP2 gene virus-like particles in mice Immunology 2014 May; 142 (1): 58-66.doi: 10.1111/imm.12202).

Thus, suitable candidate isolated bioactive respiratory virus immunogens of use in the invention may be selected from one or more of the following: the MERS spike protein (MERS-S) and immunogenic parts thereof; SARS-COV-2 spike protein and immunogenic parts thereof, the trimeric spike protein and immunogenic components thereof, the SARS-COV-2 nucleocapsid protein and immunogenic parts thereof, spike protein receptor binding domain (S-RBD) and immunogenic parts thereof, the S1 subunit of the spike(S) protein and immunogenic parts thereof such as the C-domain, and the transmembrane protease TMPRSS2 and immunogenic components thereof; the influenza virus A computationally optimized broadly reactive antigen (COBRA) neuraminidase (NA) surface protein, (i.e. the N1-1 COBRA NA antigen), haemagglutinin antigens (HA) for the variable head domain of HA, the HA stalk structure, composed of portions of HA1 and all of HA2, headless HA, chimeric HA, mosaic HA, computationally-optimized broadly reactive antigens (COBRA), and “breathing” HA; the respiratory syncytial virus (RSV) G and F glycoproteins proteins from RSV A and RSV B such as PFP-1 and/or PFP-2 and/or PFP-3 a, co-purified F, G and matrix (M) proteins, and BBG2Na, a peptide from the G glycoprotein central conserved region of the G glycoprotein, a prokaryotically expressed fusion protein that consists of the central conserved region of the G glycoprotein from the RSV A Long strain (residues 130-230) fused to the albumin-binding domain of streptococcal protein G; the parainfluenza virus (PIV) proteins F and N of human PIV-3; the human metapneumovirus (HMPV) proteins such as protein F; rhinovirus (RV) proteins such as VP1, and VP3 from RV14 and conserved regions of VP4, RV polyprotein encompassing VP4 and VP2 (known as VP0), and RV16 VP0-VP0, and the Nlm-II region of VP2; and human bocavirus (HBoV) recombinant protein HBoV viral capsid protein 2-virus like particles.

Candidate bioactive isolated respiratory virus immunogens of use in the invention as described herein may include independent monomers, dimers, or trimers thereof, such as, trimeric RBD of the SARS-COV-2 virus. Such monomers may be fused together with linkers, such as serine linkers comprising 6 or more serine amino acid residues or S₃N₁₀ and the like. One or more different isolated bioactive respiratory virus immunogens may be presented in compositions of the invention either separately or may be fused together via a linker to generate combined vaccine immunogens against two or more diseases at the same time, such as Covid-19 and Influenza. Preferably, combinations of different antigens that may be employed in compositions of the invention are provided in a free state, that is to say, an isolated state and are not fused together using linkers. Such antigens employed in compositions of the invention may be selected from one or more strains of the same virus species and thus provide protection against more than one virus strain, so providing protection against viral mutants of a single species; or selected from viral mutants of two or more different viral species; or selected from two or more viral mutants from one species and a single mutant from a different viral species, depending on design. Thus, enhanced, and cross-protective immunity against different disease causing viruses, for example, both influenza and SARS-COV-2 may be realised, for example, by intranasal vaccination with compositions of the invention containing one or more isolated viral antigens or immunogens selected from or derived from one or more viruses, such as a subunit protein of an influenza virus, for example haemagglutinin (HA), and S-RBD of SARS-CoV-2. In one alternative, a fusion protein comprising a subunit protein of an influenza virus such as haemagglutinin (HA) and S-RBD of SARS-COV-2 may be employed. Several permutations of different viral immunogens may be employed in compositions of the invention. In certain instances, the fusion of different bioactive respiratory virus immunogens from the list described herein may be possible.

For the purposes of the present invention a divalent, multivalent, or polyvalent vaccine is one that carries a combination of antigens (immunogens) either in a free state or linked together from different strains (serotypes/serogroups) of one respiratory virus pathogen in a single composition of the invention for immunising a person or patient in need thereof against a viral respiratory disease or viral diseases, depending on design. Again, for the purposes of the present invention, a multi-disease or multi-pathogen vaccine is one that carries at least two key protective respiratory viral antigens (immunogens) in a single vaccine composition of the invention to immunise against more than one strain of a species of virus or to immunise against more than one species of a respiratory virus.

Suitable bioactive respiratory virus immunogens of use in the invention may be selected from immunogenic viral proteins of a coronavirus species selected from SARS-COV, MERS-COV, SARS-COV-2, HCov-NL63, HCov-OC43, CoV-HKU1, HCov-229E and mutant strains thereof; and from immunogenic viral proteins of an influenza A virus species selected from H1N1, H5N1, and H3N2 and mutant strains thereof. Examples of especially useful bioactive respiratory virus immunogens in the invention are selected from the SARS-COV-2 spike protein(S), the spike protein receptor binding domain (S-RBD), and immunological fragments thereof. Preferably, the said at least one bioactive respiratory virus immunogen of use in the invention is the spike protein receptor binding domain (S-RBD) and immunological fragments thereof.

Nasal mucosal cells may be placed in contact with powdered compositions of the invention containing an hygroscopic gel-forming component as herein defined, such as those including HPMC and/or carrageenan particles, the said particles preferably containing a single species of particle such as HPMC that then form an expansive gel on contact with moisture from such cells. The gel is physically overlaid on the nasal mucosal cell lining and thereby substantially covers it. Similarly, where the compositions of the invention are applied as a sprayed gel containing HPMC, it is also physically overlaid on the nasal mucosal cell lining and thereby substantially covers it. Respiratory viruses, such as SARS-COV-2 and/or influenza viruses are physically blocked or prevented from infecting mucosal cells with which the vaccine composition comes into contact and substantially slows down respiratory virus release from respiratory virus-infected mucosal cells while concomitantly reducing or substantially eliminating viral shedding from the nasal mucosa. Simultaneously, the introduced at least one isolated respiratory virus immunogen diffuses through the gel to the nasal mucosa cell lining.

Furthermore, the gel within the nasal cavity is able to hold relatively large amounts of immunogen that is capable of travelling through the gel and to the mucosal cell lining over a period of from 12 to 24 hours. The advantage of this property is that the immunogen stays out of reach of degrading biological agents, such as proteolytic enzymes for a relatively long time, and acts as a depot for the immunogen which is thus exposed to the mucosal cell lining over a sustained period of time. The flow of vaccine components, such as the immunogen, and adjuvant from the gel to the nasal mucosa is thought likely to give rise to a stronger immune response involving IgA and IgG production than immunogens introduced into the systemic circulation through conventionally applied routes, such as parenteral, subcutaneous, or intramuscular routes. It is also considered that the physical nature of the gels formed from compositions of the invention also substantially prevents the rapid loss of immunogen from the nasal cavity, for example, egress from the nasal cavity into the throat or from further egress from the nasal cavity and out of the nostril opening into the external world.

Thus, hygroscopic gel-forming components, such as carrageenan and/or HPMC-containing vaccine compositions of the invention when administered to the nasal cavity also substantially or completely blocks the release of respiratory viruses from nasal mucosal cells previously infected with viruses, such as SARS-COV-2.

The hygroscopic gel-forming material is typically made up of HPMC particles as herein described, and at least one viral immunogen/antigen. For the purposes of the present invention the terms ‘immunogen’ and ‘antigen’ are used interchangeably herein and mean the same thing unless context demands otherwise. Thus, an antigen or immunogen is an element of a vaccine that typically consists of or comprises a protein or carbohydrate derived from the pathogen or pathogens of interest against which an adaptive immune response is generated. The antigen or immunogen may be supplied in compositions of the invention as described herein as isolated immunogen per se.

Where the gel-forming component of the invention is made up of HPMC particles, such particles can be of any size provided always that when admixed with other components, such as at least one isolated immunogen, the resultant mixture possesses a mean viscosity within the range 22 Pa·s+/−2 Pa·S to 40 Pa·s.+/−5 Pa·S at 20° C. in a 3.6% aqueous solution, such as 26+/−2 Pa·S to 34+/−0.5 Pa·S., or 32 Pa·S+/−5 Pa·S at 20° C. in a 3.6% aqueous solution or the like. In a preferment, the hygroscopic gel-forming material is HPMC wherein the mean viscosity of the dry powder HPMC component per se of the invention lies within the range 10 Pa·S to 20 Pa·s. at 20° C. in a 2% aqueous solution, such as 10 to 17 Pa·S. 20° C. in a 2% aqueous solution or in the range 12 Pa·S to 15 Pa·S. The viscosities of compositions of the invention are measured using standard procedures as taught in the European Pharmacopeia Chapter 2.2.10 on Rheology Analysis and may be performed on a TA Discovery Hybrid Rheometer 1 (TA DHR1) from TA Instruments Inc., Wilmington, USA), or comparable Rheometer, and the mean viscosity of several samples calculated therefrom. A suitable mean particle size for HPMC particles of use in the invention has been found to be from 60 μm to 140 μm, although it is to be understood that the mean particle size of HPMC particles may also lie outside of this range, for example, between about 50 μm and about 150 μm as long as the vaccine composition of the invention has a mean viscosity as defined herein.

The compositions of the present invention are designed for application to the nasal mucosa through insufflation via the nose.

Compositions of the invention must be able to form gels on contact with moisture. The compositions of the invention should not contain additives that may or could substantially interfere with their ability to form gels on contact with moisture, such as additives that can significantly lower the pH of the nasal mucosa. On contact with the nasal mucosa, the dry powder particles of the invention absorb moisture and thereby form a gel matrix on the surface thereof. The function of the gel is considered to be at least twofold: firstly, it acts as a physical barrier to the uptake of viruses through the nasal mucosa and secondly it physically blocks transfection between cells. During the hydration of dry powdered compositions of the invention a gel matrix is formed through contact with moisture in the nasal passages in which larger particles and smaller particles of the gel matrix combine to form a molecular net or molecular matrix wherein the smaller particles occupy spaces or gaps between larger particles and so contribute to gel formation, helping the larger particles to subsume together more easily. Whole viral particles become trapped in the gel and are substantially unable to infect cells of the nasal mucosa. A third function of the gel-forming component of compositions of the invention, such as HPMC and carrageenan, is to act as a carrier for isolated immunogen(s) and added adjuvants to the nasal mucosa. Compositions of the invention also act to prevent immunogen and other components such as aerial born viruses from getting to the lungs. Naturally, the skilled addressee will appreciate that a fourth function of the gel-forming component is as a depot for such additives as described herein.

Respiratory virus vaccine compositions of the invention require the presence of adjuvant. The term ‘adjuvant’ and ‘an adjuvant’ when used in the context of the present invention is employed to describe either a single adjuvant species, or depending on context, at least two or more adjuvant species that are added to compositions of the invention. An adjuvant is a substance that may be added to a vaccine composition prior to presentation of the other vaccine composition components to a recipient, or together with other vaccine composition components, for example, mixed together, or as a separate component applied after the mixed immunogen and hygroscopic gel-forming material has been presented to the recipient. The adjuvant is added to stimulate and enhance the magnitude and durability of the immune response in the recipient. One or more adjuvants may be added, depending on design.

Suitable candidate adjuvants for use in compositions of the invention may be selected from R848 Vaccigrade™, alum salts, such as potassium aluminium sulphate, aluminium hydroxide, aluminium phosphate, ASO4 (3-O-desacyl-4′-monophosphoryl lipid A [MPL] adsorbed on alum), ASO3 (squalene oil-in-water emulsion containing α-tocopherol [vitamin E], ASO1 (TLR4 ligand and a purified saponin fraction [QS-21, a triterpene glycoside] are formulated together in liposomes in the presence of cholesterol), MF59 (oil-in-water emulsion formed with squalene oil stabilised in aqueous buffer by non-ionic surfactants Tween 80 and Span 85), cytosine phosphoguanosine 1018 (CpG 1018: a TLR9 22-mer unmethylated CpG-B class single stranded oligonucleotide), and Matrix-M™ (Novavax proprietary adjuvant Matrix-M™, a saponin-based adjuvant consisting of two populations of individually formed 40 nm sized Matrix particles, each with a different and well characterized saponin fraction with complementary properties (Fraction-A and Fraction-C, respectively). Suitable proportions in Matrix-M are 85% Matrix-A and 15% Matrix-C). The Matrix particles are formed by formulating purified saponin from Q. saponaria Molina with cholesterol and phospholipid; and Toll-like receptor 7/Toll-like receptor 8 (TLR7/TLR8) ligand adsorbed in alum. Preferably, the adjuvant is selected from an alum salt, R848 Vaccigrade™, MF59, CpG 1018, and Matrix-M™ More preferably, the adjuvant is selected from an alum salt, and R848 Vaccigrade™, and most typically from an alum salt powder, MF59 in liquid of dry powder form (lyophilizate or freeze-dried), CpG 1018 in liquid or dry form, and R848 Vaccigrade™ in lyophilised form. The adjuvant may comprise or consist of more than one adjuvant, such as a combination selected from one or more of R848 Vaccigrade™, MF59 or CpG 1018. The selection of adjuvant should be such so as to maximise the immune response in a recipient without substantial deleterious effect on the recipient's health. In a preferred form the adjuvant may be in a lyophilised or freeze-dried powder state and is mixed with powdered hygroscopic powder, such as HPMC, and powdered immunogen in a single composition prior to application to the nasal passages.

Typical amounts of adjuvant that may be added to compositions of the invention range up to 100 μg per dose of compositions of the invention, depending on viral disease to be immunised against. Suitable amounts of adjuvant may lie in the range of ≥2.0 μg to ≤80 μg, such as from ≥2.0 μg to ≤60 μg, or ≥2.0 μg to ≤40 μg per dose or any amount therein between.

Respiratory virus vaccine compositions of the invention can be employed on mammals, such as ones selected from a mouse, a hamster, a rat, a ferret, a horse, a camel, an ape, a monkey, and a human being.

Preferably, a respiratory virus vaccine composition of the invention for nasal administration to a mammal, such as a human being, consists of dry powdered gel-forming hygroscopic material capable of forming a gel in situ (i.e. on nasal mucosal tissue), such as carrageenan or hydroxypropyl methylcellulose (HPMC) or a mixture thereof. Preferably, the powdered hygroscopic material consists solely of HPMC, at least one bioactive respiratory virus immunogen as defined herein, and an adjuvant as defined herein. Where the vaccine composition is in a dry, particulate form, the mix incorporates HPMC particles of sizes as defined herein.

In a further aspect of the invention there is provided a respiratory virus vaccine composition for nasal administration to a mammal, such as a human being, comprising hydroxypropyl methylcellulose (HPMC), at least one bioactive respiratory virus immunogen, and an adjuvant wherein the said composition is in the form of a powder or gel comprising hydroxypropyl methylcellulose (HPMC) as defined herein, at least one bioactive respiratory virus immunogen as defined herein, and an adjuvant as defined herein, and physiological saline or a phosphate-buffered saline.

In a further aspect of the invention, respiratory virus vaccine compositions of the invention may also be supplied in a two part form for nasal administration to a mammal, such as a human being, comprising:
a first formulation comprising a gel-forming hygroscopic material, such as hydroxypropyl methylcellulose (HPMC) particles as defined herein and at least one isolated bioactive respiratory virus immunogen as defined herein or an immunological fragment thereof; and
a second formulation comprising an adjuvant as defined herein, wherein the first formulation and second formulation are administered together and/or sequentially to a recipient mammal, such as a human being via the nasal route.

The first formulation in this aspect of the invention may be administered first, followed immediately by the second formulation or the second formulation may be administered first followed immediately by the first formulation.

Preferably, respiratory virus vaccine compositions of the invention may also be supplied in a two part form for nasal administration to a mammal, such as a human being, consisting of:
a first formulation consisting of a gel-forming hygroscopic material, such as hydroxypropyl methylcellulose (HPMC) particles as defined herein and at least one bioactive respiratory virus immunogen as defined herein or an immunological fragment thereof; and
a second formulation consisting of an adjuvant as defined herein, wherein the first formulation and second formulation are administered together and/or sequentially to a recipient via the nasal route.

In a preferment of this aspect of the invention, the first formulation may be administered first, followed immediately by the second formulation, or the second formulation may be administered first followed immediately by the first formulation.

In a further aspect of the invention there is provided a kit comprising the vaccine composition components as defined herein, wherein the HPMC particles are held in a first receptacle; the respiratory virus immunogen is held in a second receptacle; and the adjuvant is held in a third receptacle, wherein all three receptacles are substantially airtight; and optionally, a fourth empty receptacle. The empty receptacle can be filled with all three vaccine components and thoroughly mixed therein before being used to deliver a dose of the complete mixed vaccine composition to the nasal passages of a recipient through a nozzle.

Compositions of the invention and components thereof can be dispensed from receptacles as disclosed in, for example, EP1368090B1 and EP3183022B1, the teaching of which is incorporated herein in its entirety. The receptacles disclosed therein use a very simple mechanism for restricting the amount of content which is dispensed thus ensuring that enough immunogen is applied to the nasal mucosa in the form of a plume of powder and further, that blockage of the nasal tract is unlikely to occur.

Each of the receptacles of use in the invention typically comprise deformable and resilient surfaces that are depressible with the first finger and thumb so as to permit the administration of powder to the nasal mucosa. Preferably still, the fourth empty receptacle, if present, also comprises deformable and resilient sides that are depressible with the finger and thumb so as to permit the administration of powder therefrom to the nasal mucosa of a recipient.

The respiratory virus vaccine composition components as herein described may be first mixed and then applied to the nasal mucosa or each component may be administered sequentially as described above. In either method of administration, the receptacle of choice for administering each powder whether mixed or independently administered can be a squeezy bottle as defined and described in either of EP1368090B1 and EP3183022B1.

In a further variant of this aspect of the invention, there is provided a kit comprising the respiratory virus vaccine composition components as defined herein, wherein the HPMC particles and the at least one respiratory virus immunogen are in a dry powder mixture and held in a first receptacle; and the adjuvant in lyophilised form or in a liquid form is held in a second receptacle, wherein both the first and second receptacles are substantially airtight. The components may be added together and mixed in one or other of the two receptacles or optionally, may be placed in a third receptacle and mixed therein.

In a still further aspect of the invention there is provided a receptacle containing a respiratory virus vaccine composition comprising HPMC particles, the at least one respiratory virus immunogen and the adjuvant in a substantially dry powder mixture, wherein the vaccine composition components are as defined herein, and wherein the receptacle is substantially airtight.

In a variant of this aspect of the invention there is provided a receptacle containing a respiratory virus vaccine composition comprising HPMC particles, the at least one respiratory virus immunogen in lyophilised form and an adjuvant in lyophilised form in a substantially dry powder admixture, wherein the vaccine composition components are as defined herein, and wherein the receptacle is substantially airtight.

In a further variant of this aspect of the invention there is provided a receptacle containing a respiratory virus vaccine composition for nasal administration to a mammal, such as a human being, wherein the said composition is in the form of a gel comprising hydroxypropyl methylcellulose (HPMC) as defined herein, at least one bioactive respiratory virus immunogen as defined herein, and an adjuvant as defined herein, and physiological saline, and wherein the said receptacle is substantially airtight.

In a still further variant of this aspect of the invention, there is provided a receptacle containing a respiratory virus vaccine composition for nasal administration to a mammal, such as a human being, wherein the said composition is in the form of a gel consisting of hydroxypropyl methylcellulose (HPMC) as defined herein, at least one bioactive respiratory virus immunogen as defined herein, and an adjuvant as defined herein, and physiological saline, and wherein the said receptacle is substantially airtight.

Naturally, the skilled addressee will appreciate that where a physiological saline is employed in compositions of the invention it may be a simple physiological saline or it may be a physiologically acceptable buffered saline, such as a phosphate buffered saline.

Respiratory virus vaccine compositions according to the present invention may further comprise excipients commonly employed in vaccine formulations, such as a selection from the following: 1,2-distearoyl-sn-glycero-3-phophocholine [DSPC], acetic acid, albumin (Ovalbumin), albumin (fetal bovine serum), amino acid (arginine), β-Propiolactone, calcium carbonate, calcium chloride, protein purifiers such as cetyltrimethylammonium bromide (CTAB), cholesterol, citric acid monohydrate, D-glucose, DNA, preservative such as EDTA, egg protein (ovalbumin), Ferric (III) nitrate, formaldehyde, hydrolysed porcine gelatin, antimicrobials such as gentamycin sulphate, neomycin, neomycin sulphate, polymyxin, polymyxin Band kanamycin, hydrocortisone, L-cystine, L-tyrosine, magnesium sulphate, monosodium glutamate, surfactants such as sodium deoxycholate, sodium taurodeoxycholate, Nonylphenol Ethoxylate, Octoxynol-10 (Triton X-100), Octylphenol Ethoxylate, polysorbate 20, polysorbate 80, pH/dyes such as phenol red indicator, buffers such as phosphate buffer, potassium chloride, sodium citrate, polyethylene glycols such as (SM-102, polyethylene glycol [PEG], 2000 dimyristoyl glycerol [DMG], 2[(polyethylene glycol)-2000-N,N-ditetradecylacetamide; common salt, stabilisers/solvents such as sorbitol, sucrose, preservatives such as thimerosal, vitamins and any combination thereof. The man skilled in the art is well acquainted with the kinds of excipients that may be employed in respiratory virus compositions (see www.vaccinesafety.edu), the above being but a selection of excipients employed in vaccine formulations at this time. It is to be understood that the inclusion of excipients into compositions of the invention should be such so as not to substantially interfere with the gel-forming properties of the hygroscopic component of the invention and its' at least three functions as outlined herein.

In preferred embodiments of the present invention, the powder compositions do not include components which are often used in intranasal compositions (dry powders or solutions) which can cause irritation or affect ciliary movement, for example, solvents, absorption enhancers, such as cyclodextrins or glycosides, or muco-adhesives such as chitosan. The use of such additives can be undesirable, as they can cause discomfort and interfere with the normal functioning of the nose, which can adversely affect breathing.

In a further aspect of the invention there is provided a respiratory virus vaccine composition for nasal administration to a mammal as defined herein, wherein the said composition is in the form of a gel comprising a mix of a gel-forming hygroscopic material, such as hydroxypropyl methylcellulose (HPMC) as defined herein, at least one isolated bioactive respiratory virus immunogen as defined herein, and an adjuvant as defined herein, and physiological saline, for use in prophylaxis against respiratory viral disease caused by respiratory viruses as defined herein.

In a further aspect of the invention there is provided a respiratory virus vaccine composition for nasal administration to a mammal as defined herein, wherein the said composition is in the form of a gel consisting of a mix of a gel-forming hygroscopic material, such as hydroxypropyl methylcellulose (HPMC) as defined herein, at least one isolated bioactive respiratory virus immunogen as defined herein, and an adjuvant as defined herein, and physiological saline, for use in prophylaxis against respiratory viral disease caused by respiratory viruses as defined herein.

In a still further variant of this aspect of the invention, there is provided a receptacle containing a respiratory virus vaccine composition for nasal administration to a mammal as defined herein, wherein the said composition is in the form of a gel-forming hygroscopic material comprising hydroxypropyl methylcellulose (HPMC) as defined herein, at least one isolated bioactive respiratory virus immunogen as defined herein, and an adjuvant as defined herein, and physiological saline, for use in prophylaxis against respiratory viral disease caused by respiratory viruses as defined herein.

In a still further aspect of the presentation there is provided use of a respiratory virus vaccine composition for nasal administration to a mammal as defined herein, wherein the said composition is in the form of a gel comprising hydroxypropyl methylcellulose (HPMC) as defined herein, at least one bioactive respiratory virus immunogen as defined herein, and an adjuvant as defined herein, and physiological saline, in a method of vaccinating a mammal.

In a still further variant of this aspect of the invention, there is provided use of a respiratory virus vaccine composition for nasal administration to a mammal as defined herein, wherein the said composition is in the form of a gel consisting of hydroxypropyl methylcellulose (HPMC) as defined herein, at least one bioactive respiratory virus immunogen as defined herein, and an adjuvant as defined herein, and physiological saline, in a method of vaccinating a mammal.

In a still further aspect of the present invention there is provided a method of making a respiratory virus vaccine composition as defined herein comprising:
1) adding lyophilised viral immunogen as defined herein to hydroxypropyl methylcellulose powder as defined herein at a temperature≥10° to ≤18° C.;
2) gently blending the two ingredients of 1) in a blending machine; and
3) adding lyophilised adjuvant as defined herein and further blending.

Dry powder ingredients may be blended together using a ribbon blender, or similar type of blender for approximately 15 to 20 minutes. The time of mixing is dependent upon the moisture content and compatibility of the powders. Ingredients preferably have a moisture content of less than 5% immediately after blending as checked with the United States Pharmacopeia and National Formulary (USP/NF) loss on drying method.

In a still further variant of this aspect of the present invention there is provided a method of making a respiratory virus vaccine composition in the form of a gel as defined herein comprising:
1) adding lyophilised immunogen as defined herein to hydroxypropyl methylcellulose powder as defined herein at a temperature≥10° to ≤18° C.;
2) gently blending the two ingredients of 1) in a blending machine; and
3) adding an adjuvant as defined herein and further blending;
4) adding physiological saline to the blended product of step 3).

As a further aspect of the invention, there is provided a respiratory virus vaccine composition as defined herein that comprises or consists of from 80% w/w to 95% w/w HPMC, 2% to 10% w/w, preferably from 2% to 5% w/w, of a respiratory virus immunogen as defined herein and up to 10% w/w of adjuvant. Preferably, there is provided a respiratory virus vaccine composition of the invention as defined herein that comprises or consists of from 89% w/w to 91% w/w, preferably 90% w/w HPMC of the total weight of the said composition.

In a yet further aspect of the present invention, there is provided use of a respiratory virus vaccine composition as defined herein, use of a kit as defined herein, and/or use of a receptacle as defined herein, in the prophylactic treatment of Covid-19 disease.

In a yet further aspect of the present invention there is provided use of a respiratory virus vaccine composition as defined herein in a method of boosting or further potentiating an immunological response in a mammal that has had at least a first prior vaccination event.

The first prior vaccination event may be with a respiratory virus vaccine composition of the present invention or with a vaccine composition different from that of the present invention and made by a competitor. The first vaccination event when using a respiratory virus vaccine composition different to that of the present invention may have been given by injection, such as by an intramuscular, parenteral, or subcutaneous route or through an oral route or through a nasal route. Such a first vaccination event may have delivered a vaccine composition including a protein immunogen and/or a carbohydrate immunogen, or it may have employed a DNA vector or an mRNA vector, such as one or more of those referred to herein, for example, the sputnik V adenoviral vectors (both the injectably applied and nasally applied forms), the Moderna vaccine vectors, the Astrazeneca viral constructs and the like. Such first vaccination events elicit a systemic IgG response and a broad repertoire of central memory B and T cells. The respiratory virus vaccine compositions of the present invention may be used as an intranasal booster that recruits memory B and T cells to the nasal passages and further guides their differentiation toward mucosal protection, including IgA secretion and tissue-resident memory cells in the respiratory tract. Respiratory virus vaccine compositions of the invention are thought to be safer, less costly, and simpler to administer than competitor vaccine compositions containing mRNA and/or DNA vectors and as such will have clear advantages used as primary boosters for many of the vaccine formulations known in the art.

Thus, novel elements as described hereinabove of the present invention include the following:
i) An inventive respiratory virus vaccine composition for nasal administration to a mammal, such as a mouse, a hamster, a rat, a ferret, an ape, a monkey, a horse, a camel, and a human being comprising an hygroscopic gel-forming material, at least one isolated bioactive respiratory virus immunogen, and an adjuvant. The hygroscopic gel-forming material is selected from natural polymers such as starch, collagen, and lecithin, for example, in the form of lecithin in stabilised polymeric micelles, carrageenan and hydroxypropylmethyl cellulose. A preferred hygroscopic material is selected from carrageenan and hydroxypropylmethyl cellulose, and most preferred is hydroxypropylmethyl cellulose.
ii) The inventive composition is in the form of a dry particulate powder, the said composition being in the form of particles having a mean particle size diameter of ≥10 μm to ≤400 μm, ≥50 μm to ≤300 μm, ≥50 μm to ≤150 μm, ≥60 μm to ≤140 μm, or ≥80 μm to ≤120 μm and wherein the said immunogen is at least one viral protein in lyophilised form.
iii) The inventive composition can be in the form of a dry particulate powder comprising or consisting of:
i) dry powder hygroscopic gel-forming material particles; and
ii) at least one isolated bioactive respiratory virus immunogen; and
iii) an adjuvant,
   wherein the said respiratory virus vaccine composition has a mean viscosity within the range 22 Pa·s+/−2 Pa·S to 40 Pa·s.+/−5 Pa·S at 20° C. in a 3.6% aqueous solution, such as a range 26+/−2 Pa·S to 34+/−0.5 Pa·S. at 20° C. in a 3.6% aqueous solution.
iv) The inventive composition includes at least one respiratory virus immunogen selected from isolated immunogenic viral proteins of viruses selected from the group: coronaviruses, influenza viruses such as strains of influenza A, parainfluenza viruses, metapneumoviruses, respiratory syncytial viruses, rhinoviruses and bocaviruses. Suitable isolated bioactive respiratory virus immunogens are selected from isolated immunogenic viral proteins of a coronavirus species selected from SARS-COV, MERS-CoV, SARS-COV-2, HCov-NL63, HCov-OC43, CoV-HKU1, HCov-229E and mutant strains thereof; and from immunogenic viral proteins of an influenza A virus species selected from H1N1, H5N1, and H3N2 and mutant strains thereof. Isolated bioactive respiratory virus immunogenic viral proteins of use in the invention are selected from the MERS spike protein (MERS-S) and immunogenic parts thereof, SARS-COV-2 spike protein and immunogenic parts thereof, the trimeric spike protein and immunogenic parts thereof, the SARS-COV-2 nucleocapsid protein and immunogenic parts thereof, spike protein receptor binding domain (S-RBD) and immunogenic parts thereof, the S1 subunit of the spike(S) protein and immunogenic parts thereof such as the C-domain, and the transmembrane protease TMPRSS2 and immunogenic components thereof; the influenza virus A computationally optimized broadly reactive antigen (COBRA) neuraminidase (NA) surface protein, (i.e. the N1-I COBRA NA antigen), haemagglutinin antigens (HA) for the variable head domain of HA, the HA stalk structure, composed of portions of HA1 and all of HA2, headless HA, chimeric HA, mosaic HA, computationally-optimized broadly reactive antigens (COBRA), and “breathing” HA; the respiratory syncytial virus (RSV) G and F glycoproteins proteins from RSV A and RSV B such as PFP-1 and/or PFP-2 and/or PFP-3 a, co-purified F, G and matrix (M) proteins, and BBG2Na, a peptide from the G glycoprotein central conserved region of the G glycoprotein, a prokaryotically expressed fusion protein that consists of the central conserved region of the G glycoprotein from the RSV A Long strain (residues 130-230) fused to the albumin-binding domain of streptococcal protein G; the parainfluenza virus (PIV) proteins F and N proteins of human PIV-3; the human metapneumovirus (HMPV) proteins such as protein F; rhinovirus (RV) proteins such as VP1, and VP3 from RV14 and conserved regions of VP4, RV polyprotein encompassing VP4 and VP2 (known as VP0), and RV16 VP0-VP0, and the Nlm-II region of VP2; and human bocavirus (HBoV) recombinant protein HBoV viral capsid protein 2-virus like particles. Suitable virus immunogens of use in the invention include the SARS-COV-2 spike protein(S), the spike protein receptor binding domain (S-RBD), and immunological fragments thereof.

v) The adjuvant is selected from one or more of R848 Vaccigrade™, alum salts, such as potassium aluminium sulphate, aluminium hydroxide, aluminium phosphate, ASO4 (3-O-desacyl-4′-monophosphoryl lipid A [MPL] adsorbed on alum), ASO3 (squalene oil-in-water emulsion containing α-tocopherol [vitamin E], ASO1 (TLR4 ligand and a purified saponin fraction [QS-21, a triterpene glycoside] are formulated together in liposomes in the presence of cholesterol), MF59 (oil-in-water emulsion formed squalene oil stabilised in aqueous buffer by non-ionic surfactants Tween 80 and Span 85), cytosine phosphoguanosine 1018 (CpG 1018: a TLR9 22-mer unmethylated CpG-B class single stranded oligonucleotide), Matrix-M™ (Novavax proprietary adjuvant Matrix-M™, a saponin-based adjuvant consisting of two populations of individually formed 40 nm sized Matrix particles, each with a different and well characterized saponin fraction with complementary properties (Fraction-A and Fraction-C, respectively. Suitable proportions in Matrix-M are 85% Matrix-A and 15% Matrix-C). The Matrix particles are formed by formulating purified saponin from Q. saponaria Molina with cholesterol and phospholipid; and Toll-like receptor 7/Toll-like receptor 8 (TLR7/TLR8) ligand adsorbed in alum. Preferred adjuvants of use in the inventive composition include those selected from an alum salt, R848 Vaccigrade™, MF59, CpG 1018, and Matrix-M™, those selected from an alum salt, MF 59, CpG 1018 and R848 Vaccigrade™, preferably in a dry powder form, such as a lyophilised or freeze dried form.
vi) The inventive respiratory virus vaccine composition for nasal administration to a mammal consists of an hygroscopic gel-forming material, at least one isolated bioactive respiratory virus immunogen as defined herein, and an adjuvant as defined herein. The inventive respiratory virus vaccine composition can be in the form of a gel comprising or consisting of an hygroscopic gel-forming material, at least one isolated bioactive respiratory virus immunogen as defined herein, and an adjuvant as defined herein, and physiological saline or the inventive respiratory virus vaccine composition can comprise or consists of: a first formulation comprising hygroscopic gel-forming material particles and at least one bioactive respiratory virus immunogen as defined herein or an immunological fragment thereof; and a second formulation comprising an adjuvant as defined herein, wherein the first formulation and second formulation are administered together or sequentially to a patient via the nasal route.

The inventive respiratory virus vaccine composition can consist of:
a first formulation consisting of hygroscopic gel-forming material particles and at least one bioactive respiratory virus immunogen as defined herein or an immunological fragment thereof; and
a second formulation consisting of an adjuvant as defined herein, wherein the first formulation and second formulation are administered together and/or sequentially to a patient via the nasal route.
vii) The inventive composition may be supplied in an inventive kit comprising the respiratory virus vaccine composition components as defined herein, wherein the hygroscopic gel-forming material particles are held in a first receptacle; the respiratory virus immunogen is held in a second receptacle; and the adjuvant is held in a third receptacle, wherein all three receptacles are substantially airtight. An alternative kit form comprises the respiratory virus vaccine composition components as defined herein, wherein the hygroscopic gel-forming material particles and the at least one respiratory virus immunogen are in a dry powder admixture and held in a first receptacle; and the adjuvant is held in a second receptacle, wherein both the first and second receptacles are substantially airtight.
viii) The inventive respiratory virus vaccine composition can be supplied in a receptacle containing a respiratory virus vaccine composition comprising hygroscopic gel-forming material particles, the at least one respiratory virus immunogen and the adjuvant in a substantially dry powder admixture, wherein the vaccine composition components are as defined herein, wherein the receptacle is substantially airtight. Suitably, the immunogen and adjuvant are in a lyophilised form.
   The inventive respiratory virus vaccine composition can be supplied in the form of a receptacle wherein the said composition is in the form of a gel as defined herein, wherein the receptacle is substantially airtight.
ix) The inventive respiratory virus vaccine composition as defined herein, or a kit as defined herein, or a receptacle as defined herein is for use in the prophylactic treatment of respiratory virus disease.
x) Inventive methods of making an inventive respiratory virus vaccine composition as defined herein are also includes within the spirit if the invention. One method comprises:
1) adding lyophilised immunogen as defined herein to hydroxypropyl methylcellulose powder at a temperature≥10° to ≤18° C.;
2) gently blending the two ingredients of 1) in a blending machine; and
3) adding lyophilised adjuvant as defined herein and further blending.

Another variant on the method of making a respiratory virus vaccine composition as defined herein comprises:
1) adding lyophilised immunogen as defined herein to hydroxypropyl methylcellulose powder at a temperature≥10° to ≤18° C.;
2) gently blending the two ingredients of 1) in a blending machine; and
3) adding an adjuvant as defined herein and further blending;
4) adding physiological saline to the blended product of step 3).
xi) The inventive respiratory virus vaccine composition as defined herein, wherein said composition comprises or consists of from 80% w/w to 95% w/w HPMC and 2% to 10% w/w, preferably from 2% to 5% w/w, of a respiratory virus immunogen as defined herein. A variant inventive respiratory virus vaccine composition as defined herein, is one wherein the HPMC component comprises or consists of 89% w/w to 91% w/w, preferably 90% w/w of the total weight of the composition.
xii) The inventive respiratory virus vaccine composition as defined herein, or a kit as defined herein, or a receptacle as defined herein for use in the prophylactic treatment of Covid-19 disease. As a variant, a respiratory virus vaccine composition as defined herein for use in boosting or further potentiating the treatment of a respiratory virus disease is also provided. As a further variant, also provided is a respiratory virus vaccine composition as defined herein in a method of boosting or further potentiating the treatment of an immunological response in a mammal, such as a human being, that has had at least a first prior vaccination event against a respiratory virus disease. Such inventive respiratory virus vaccine compositions as defined herein include compositions wherein the respiratory virus disease is selected from Covid-19 and influenza or is influenza or is Covid-19.
xiii) The inventive respiratory virus vaccine composition in the form of a dry particulate powder comprising:
i) dry powder hydroxypropyl methylcellulose (HPMC) particles; and
ii) at least one isolated bioactive respiratory virus immunogen; and
iii) an adjuvant,
   wherein the mean viscosity of the dry powder HPMC particles per se lies within 10 to 20 Pa·S, at 20° C. in a 2% aqueous solution, such as, 10 Pa·S to 17 Pa·S at 20° C. in a 2% aqueous solution.

There now follows examples and Figures illustrating the invention. It is to be understood that the Figures and examples are not to be construed as limiting the invention in any way.

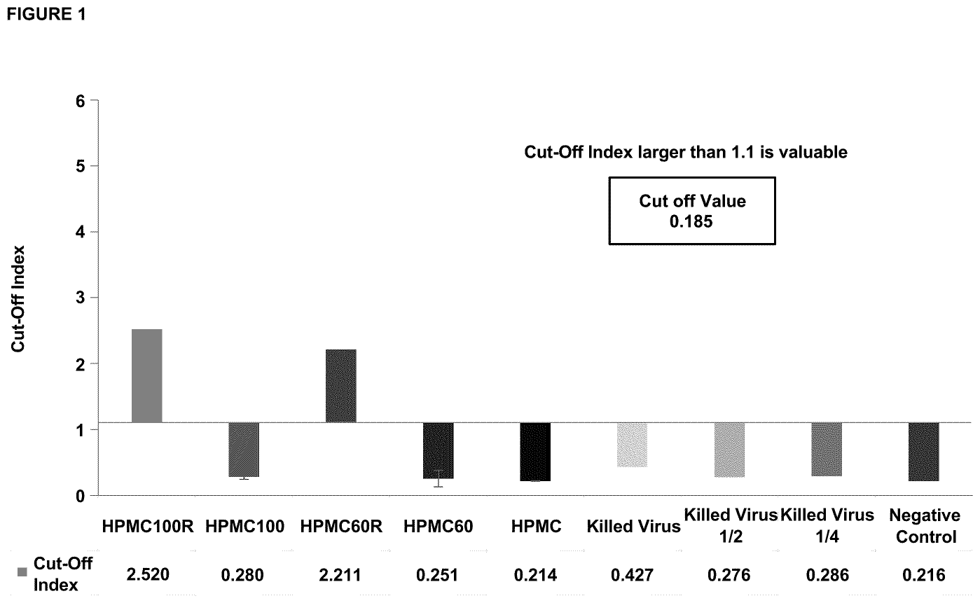

FIG. 1: Cut-off index of hamsters' serum on the 35^{th} day. The data were statistically compared to HPMC100R.

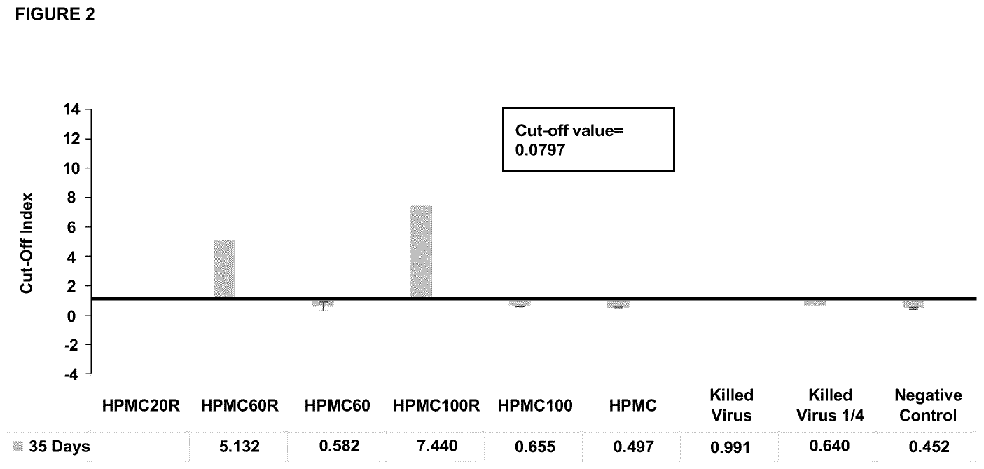

FIG. 2: Cut-off index of hamsters' serum on 35^{th} day. Cut off Value was 0.0797.

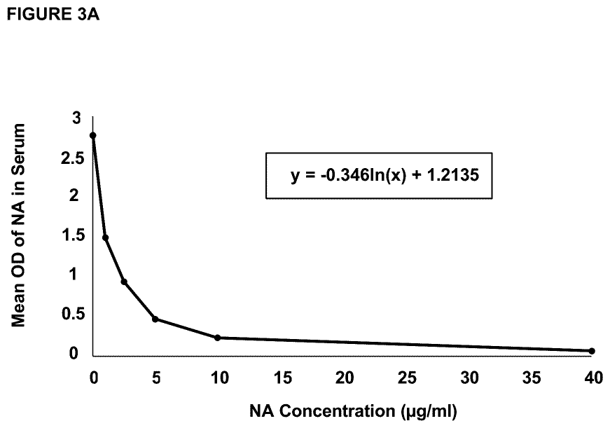

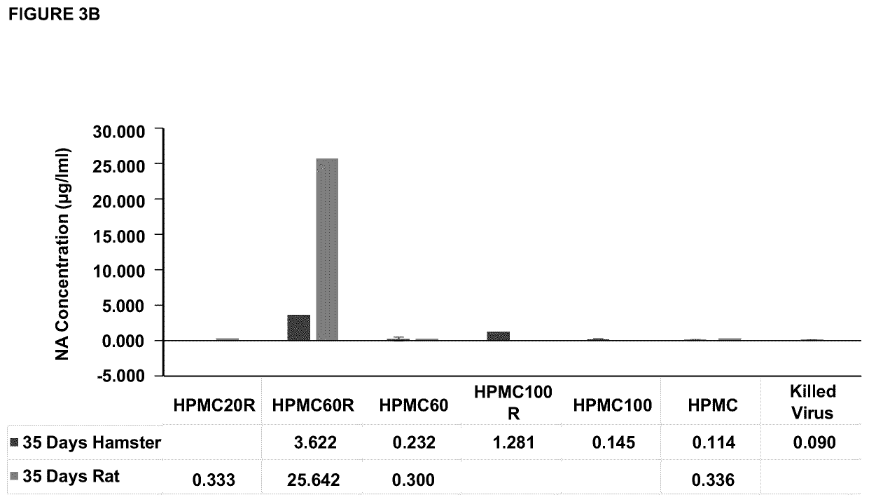

FIG. 3A: Standard curve graph.

FIG. 3B: Concentration of NA in rats on different days. Rats received 20 and 50 μg of spike solution. Rat received 30 μg adjuvant for the first dose and 15 μg for the second dose.

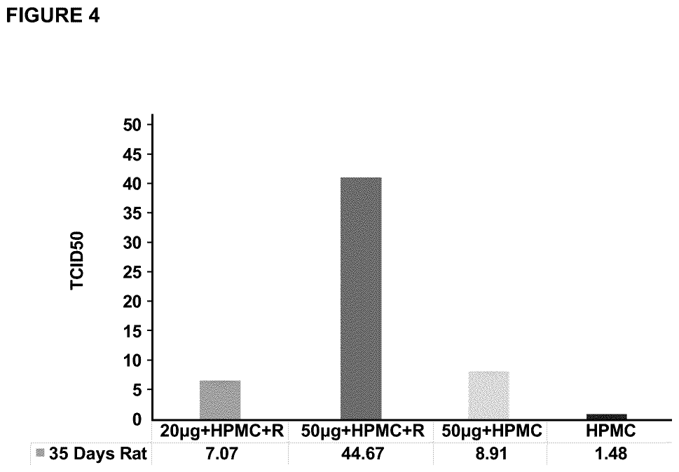

FIG. 4: TCID50 titre derived from VNA test. Rats received 20 and 50 μg of spike solution by 35 days. Rats received 30 μg of adjuvant for the first dose and 15 μg second dose.

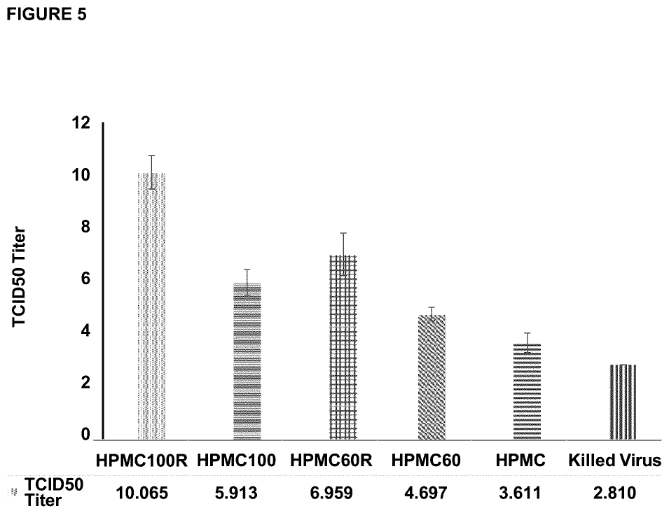

FIG. 5: TCID50 titre derived from VNA test of hamsters on the 35^{th} day.

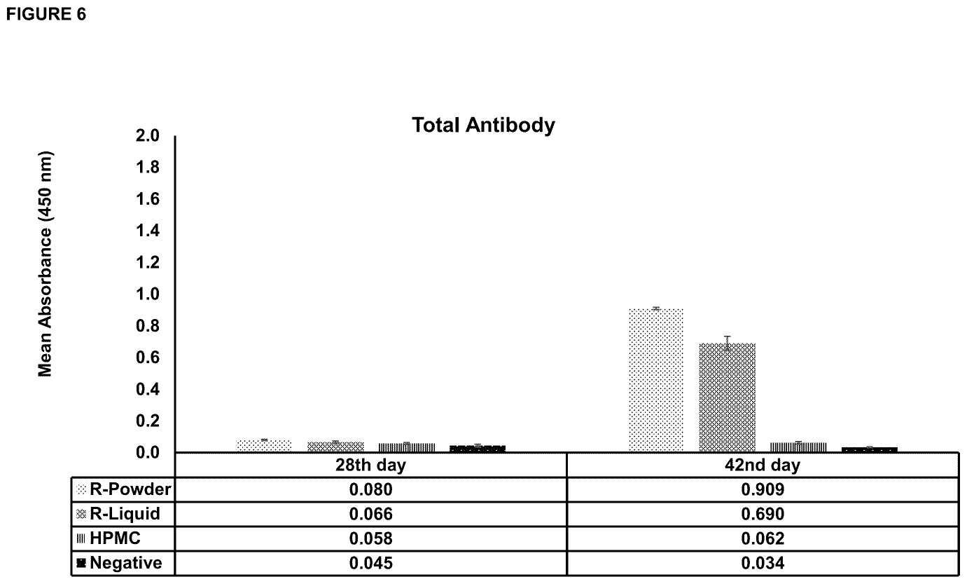

FIG. 6: Total antibodies in rabbits treated by formulations of COVID-19 vaccines. There were significantly higher antibodies on R-Powder compared with others in rabbit.

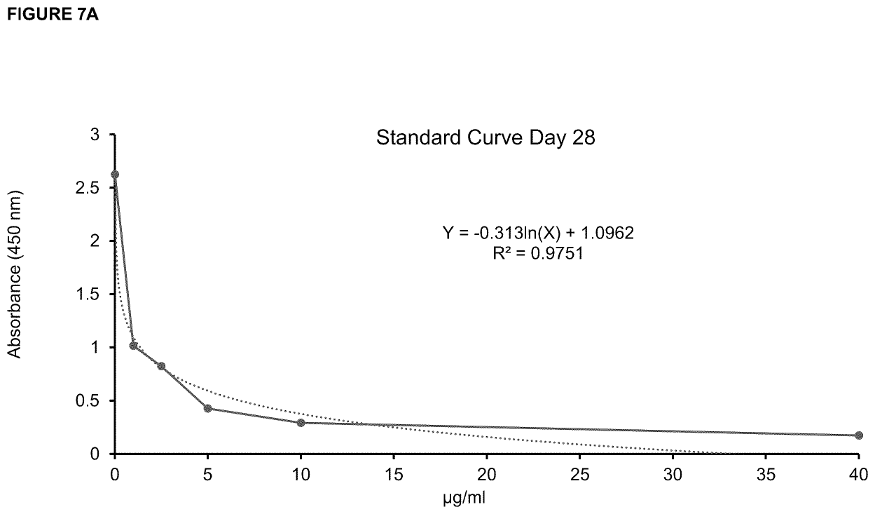

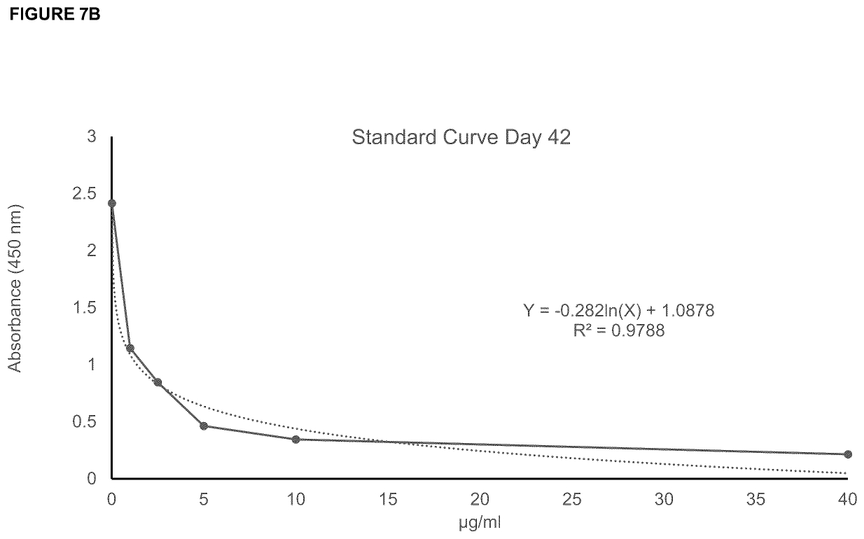

FIG. 7A: A) standard cure on 28^{th} day in rabbit.

FIG. 7B: B) standard curve on 42^{nd} day in rabbit.

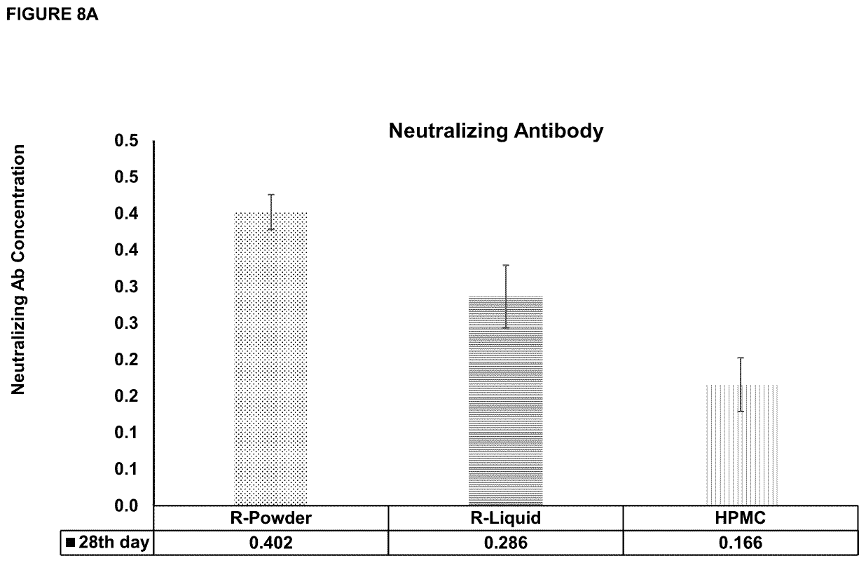

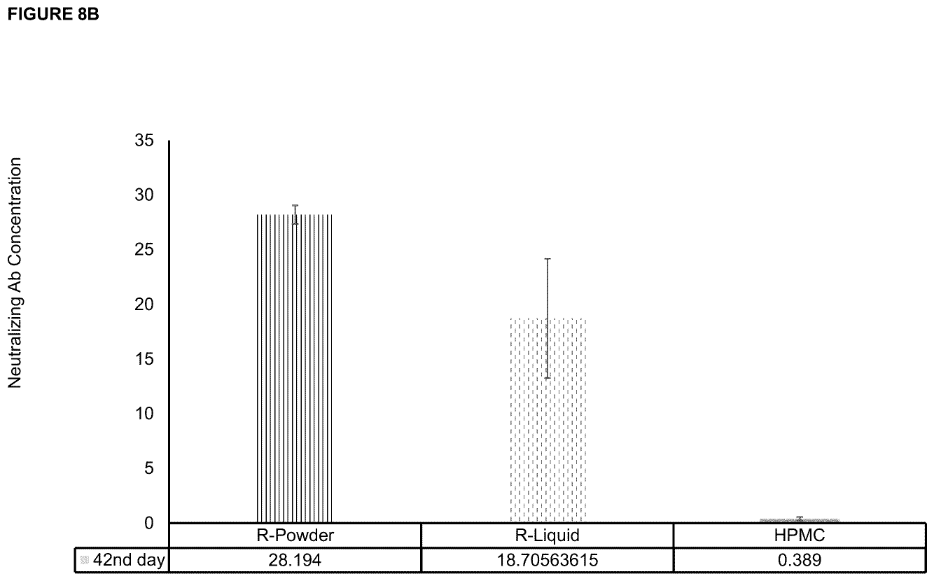

FIG. 8A: Neutralizing antibody on the 28^{th} day in rabbit.

FIG. 8B: Neutralizing antibody on the 42^{nd} day in rabbit.

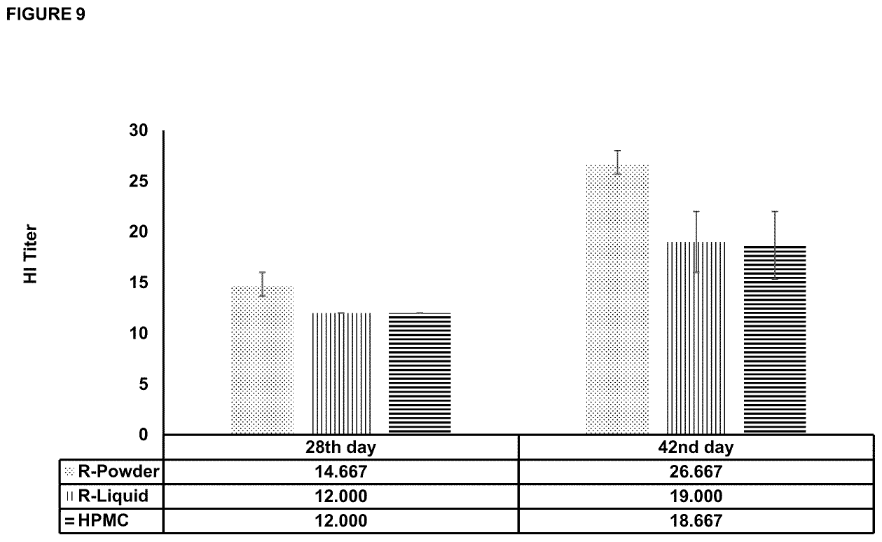

FIG. 9: HI, titre on the 28^{th} and 42^{nd} days in rabbit. There was a significant HI antibody titre in treated groups to trigger antibodies against the Influenza virus.

### EXPERIMENTAL SECTION

### Aim

This study demonstrates the development of an intranasal vaccine using hydroxypropyl methylcellulose as a delivery carrier for the receptor-binding domain (RBD) of the spike protein of SARS-COV-2 virus.

### Materials & Method

Four putative vaccine formulations (HPMC100R, HPMC100, HPMC60R, and HPMC60) were tested for possible use against SARS-COV-2. The control was HPMC. The tests were carried out using in cells culture using VERO cells and a preclinical animal model (hamster). Two techniques were used to confirm the levels of antibodies 35 days post-exposure to the vaccine formulations. These were neutralizing antibodies (NA) in serum using a commercially available NA kit and SARS-COV-2 virus in VERO cell culture.

### Results

In preclinical hamster studies, using a Cut-Off index of total antibodies in serum, showed that both the HPMC100R and HPMC60R groups had significantly higher antibody levels than control. The antibody levels in the HPMC100R group were higher than the levels measured in the HPMC60R group. That said, the difference between the HPMC60R group and that of the HPMC100R group did not appear to be statistically significant. This is probably due to the extant difficulty of getting powdered components into the nasal passages. The changes in antibody levels in the groups HPMC100 and HPMC60 were not significant opposite control.

There was no significant difference observed in temperature changes and body weight in hamsters during the entire timeline of the experiments (data not shown).

The results of the NA test in exposure to the virus in VERO cells indicated that the HMPC100R group induced a higher amount of NA than other groups. Tissue Culture Infectious Dose (TCID50) assay and the concentration of NA in hamsters treated by Nasal formulation was significantly higher than the subcutaneously injected killed virus in group 6.

Serum of rats treated by HPMC50R showed a higher titre of NA in competition with hACE2 in the NA kit (NA=25 μg/ml) and also in the face of live virus (TCID50=44).

In summary, both formulations HPMC100R and HPMC60R induced significantly higher total antibody and NA when compared with control and other formulations.

### Abbreviations

| | |
|---|---|
| hACE2 | human angiotensin converting enzyme 2 |
| TCID50 | Tissue Culture Infectious Dose assay |
| NA | Neutralizing antibody |
| SD | Standard Deviation |
| RNA | Ribonucleic acid |
| Vero cells | A cell was isolated from kidney epithelial cells extracted from |
| | an African green monkey |
| RdRP | RNA dependent RNA polymerase |
| RBD | Receptor binding domain |
| G614 | Mutation in genome |
| D614G | Substitution and mutation in genome |
| ADE | Antibody dependent enhancement |
| Y501 | Mutation in genome |
| ACE2 | Angiotensin-converting enzyme 2 |
| COI | Cut off index as a reference to illustrate a positive or negative |
| | result. The test targets antibodies including IgG, IgM and IgA |
| | against the nucleocapsid of the SARS-CoV-2 virus. |
| HMPC | Hydroxypropyl methylcellulose |

### Introduction

In 2019, the whole world came together to confront a new, life-threatening virus called “SARS-COV-2”, which causes COVID-19 disease. From 2019 until the present, new variants of the virus have emerged, and this scenario is expected to continue. The virus infects human beings through attachment to the ACE2 and CD147 receptors present in some human cells resulting in cytokine storm and death. The worse problem is a reduced efficacy of generated antibodies against other variants of SARS-COV-2. In other words, natural immunity may not work efficiently when the body faces a new variant having different exposed epitopes and several different mutations and deletions, RNA dependent RNA polymerase (RdRP) jumps, and transcription errors (1). GISAID, Nextstrain and Pango decided to use the Greek alphabet for the variant nomination of SARS-COV-2 owing to non-stigmatizing labels and for ease of pronunciation (2). Therefore, the World Health Organisation (WHO) named variants of concern based on the Greek alphabet as Alpha, Beta, Gamma, Delta, Epsilon, etc.

The transmissibility rate of the virus frequently changes. Although, it was postulated that the virulence and transmissibility of the viruses usually decreases with mutations, in this case virulence, transmissibility rate and mortality increased in some variants compared to the originally isolated Wuhan variant. Therefore, a protective strategy should be one that promises to decrease mortality, and depresses the need for hospitalization of infected persons, numbers of persons requiring intensive care unit (ICU) care and serves to prevent infection and re-infection in healthy people. To this end aim, vaccination would appear to be the best option.

Vaccines are the most efficient prophylactic formulations given to healthy people to stimulate immune responses through antibody production and cell-mediated responses. Vaccines offer the best hope of containing and eventually reversing most of the effects of covid-19 disease(s). The most important question is whether the SARS-COV-2 virus will die out soon or follow a similar path to that of the influenza virus and sporadically infect people with different endemic forms (3).

In the case of virus-based diseases, conventional methods use inactivated or live-attenuated vaccines such as Sinopharm which is an inactivated form of the original Wuhan variant cultured in Vero cells. Its efficacy is considerably less than that of the Pfizer vaccines. There were some pre-clinical SARS-COV vaccines, including recombinant S protein, vector-based, inactivated, and attenuated vaccines (4). Some of them exhibited complications in animal models; for example, the inactivated vaccines led to eosinophil infiltration in the lung and enhancement of disease (5, 6), while live vaccines led to lung damage (7).

However, there is a concern related to the severity of infection in vaccinated and re-infected COVID-19 patients which is analogous to what happened with Spanish Flu and dengue fever due to provoking the immune system, the observation of immune enhancement such as antibody dependent enhancement (ADE), and cell-based enhancement (8). Based on a study that was performed on the SARS-COV vaccine, it was shown that the vaccines against whole spike protein produced enhanced immunity whereas when the vaccine was designed against just the RBD segment of the spike protein, the protection was enhanced without immunity enhancement in animals (9). Another subject is related to the type of SARS-COV-2 variant. It is very important to choose the correct part of amino acids in a spike to design a functional spike. Some variants have similar mutations and deletions. The most popular mutations are at D614G, N501Y, 484K and 452R leading to an enhanced transmissibility (3, 10).

It has been demonstrated that spikes G614 and D614G may lead to antibody-dependent enhancement (ADE) and a high rate of spread and susceptibility to the strains with G614.

The position of 501 in the Spike protein RBD is the region where neutralizing antibodies most commonly act (11) and raises concerns about vaccine inefficiency. However, the spike deletion of 69/70 has a dual role in RBD conformational change and human immune response (12). A dynamic molecular study indicated that the N501Y mutation resulted in enhanced S1 RBD-ACE2 interaction through the hydrophobic and π-π stacking of Y501 while decreasing antibody response up to 160 times (13). Molecular dynamic results showed that K417N to N501Y mutations (B.1.351 variant) enhance the virus's binding affinity to the ACE2 receptor and decrease the binding affinity with antibodies. On the other hand, the vaccination and natural antibodies derived from earlier SARS-COV-2 variants will be less effective in protecting against the infection by the β variant, and even less effective than the α variant (14).

Most scientists focus on RBD as an important part responsible to produce neutralizing antibodies. We also used RBD of the spike protein (available from Cube Biotech, Germany) as the immunogen to stimulate the immune response and to minimise the ADE and cell-based enhancement in vaccinated people.

Additionally, adjuvants are used to enhance the immunogenicity of the desired immunogen/antigen at a lower concentration. It is noteworthy to mention that the low level of antibody induced by immunogen in individuals leads to ADE and to avoid ADE in vaccinated people, the level of antibody should be optimized. Therefore, adjuvants through enhancing the immunogenicity of antigens, overcome this issue. We used Vaccigrade R848™ as the adjuvant in our vaccine.

To deliver immunogens to the target site and to stimulate the immune response, several immunogen carrier approaches have been described. Pfizer and Moderna vaccines use nanoparticles in the form of solid lipid nanoparticles (SLN) and polyethylene glycol (PEG) as the carrier to deliver immunogen and enhanced vaccine efficacy. Other platforms use polymeric nanoparticles, virosomes, and entrapment in natural polymers. Coating and encapsulation of the immunogens on or into carriers minimizes concerns regarding safety issues and biodegradation and stability of the immunogens and enhanced immunogen bioavailability and bloodstream circulation.

Hydroxypropylmethylcellulose (HMPC) was selected in this study as the carrier to deliver immunogen and adjuvant. It is an hydrophilic, semi-synthetic, inert, visco-elastic polymer while being biocompatible and pharmaceutically safe. More complex cellulose-containing derivates have been used in preclinical vaccine studies and show promising results as a carrier for vaccine delivery (15-17). We aimed to assess the potential usefulness of HPMC powder (supplied by Dow Chemicals) with spike protein RBD protein (strain B.1.1.7 [aka the α variant] supplied by Cube Biotech), Germany) and adjuvant (Vaccigrade R848™ supplied by Invivogen, Europe) for COVID-19 infection delivered nasally to recipient animals following a design protocol supplied and largely designed by Nasaleze affiliated personnel.

In the first phase (data not shown), it was concluded that formulations with the RBD of Spike protein at a concentration of 100 μg+7.5 μg Vaccigrade R848™ induced a higher titre of protective antibodies against the live virus in hamsters. Therefore, to enhance immunogenicity and protective efficacy of the formulation, an adjuvant cocktail at a higher concentration of adjuvant and followed for a total of 35 days was performed in the rat. Results indicated in the proposed formulations with the RBD Spike at a concentration of 50 μg induced a higher titre of protective antibodies against the live virus than 20 μg.

To enhance immunogenicity and protective efficacy of the formulation in the current study, an adjuvant was added to the cocktail at a higher concentration of 30 μg and followed up over a time period of 35 days.

### Hypothesis

The cocktail of antigen, adjuvant, and HPMC powder will promote passive immunity in hamsters and will induce neutralizing antibodies which provides for protective potential against infection by SARS-COV-2.

### Research Methodology

### 1. Ethics Statement

The animal experiments were approved by the ethical committee of the Iran University of Medical Sciences (IR.IUMS.REC.1400.150). Experiments with SARS-COV-2 virus were performed at Biosafety Level-3 (BSL-3) facilities.

### 2. Vaccine Formulations

Five formulations plus one control group were prepared to evaluate total antibody and NA response in the Hamster animal model as follows:

| | | | | Adjuvant | |
|---|---|---|---|---|---|
| | | HPMC | SP (RBD) | Vaccigrade | |
| | Group | powder | Powder | (R848) ™ | |
| No. | Name | (μg) | (μg) | (μg) | Note |
| 1 | HPMC100R | 500 | 100 | 30 | 6 animals |
| 2 | HPMC100 | 500 | 100 | 0 | 6 animals |
| 3 | HPMC60R | 500 | 60 | 30 | 6 animals |
| 4 | HPMC60 | 500 | 60 | 0 | 6 animals |
| 5 | Control | 500 | 0 | 0 | 6 animals |
| 6 | Virus | — | Inacti- | 30 | Subcutaneous |
| | | | vated | | injection in |
| | | | virus | | the neck of 1 |
| | | | | | animal |

### 3. Animal Models

### 3.1. Hamster Animal Model

31 Syrian female hamsters (five groups each 6 hamsters and group 6 with one hamster were used for the evaluation of Nasaleze Vaccine formulations. All animals (3-4 months) were obtained from the animal house of Razi Vaccine and Serum Research Institute, Tehran, Iran. The animals were maintained at a controlled temperature of 24±1° C. with a 12-12 h light-dark cycle (light cycle, 07:00-19:00), and were allowed free access to water and standard hamster food ad libitum. Hamsters were randomly divided into five groups (n=6). Groups 1-5 intranasally received Nasaleze formulations on the 1^{st}, 2^{nd}, 14^{th}, and 21^{st} days while the single hamster in group 6, received a cocktail subcutaneously in the neck containing inactivated virus (TCID50: 6.5; 300 μl) plus adjuvant (30 μg R848) on the 1^{st}, 2^{nd}, 14^{th}, and 21^{st} days.

### 3.2 Rat Model

Four groups were defined to evaluate the amount of antibody protection in female rat animal models as follows; Since the amount of spike RBD available was low, the amount given to each animal was reduced from 60 μg to 50 μg and the doses were reduced from 4 to 2.
1. 20 μg SP solution+500 μg HPMC powder+adjuvant (R848 solution, 30 μg for the first dose and 15 μg the second dose) known as 20 μg
2. 50 μg SP solution+500 μg HPMC powder+adjuvant (R848 solution, 30 μg for the first dose and 15 μg the second dose) known as 60 μg
3. 50 μg SP solution+500 μg HPMC powder known as HPMC 60
4. 500 μg HPMC powder is known as HMPC

### Preclinical Evaluation

In the hamster model, the animals were followed up for 35 days. Mortality was recorded. Body temperature and body weight were monitored at 1, 2, 3, 14, 21 and 35 days in all groups.

### 1. SARS-COV-2 Antibody Kit Development and Analysis

The level of anti-SARS-COV-2 antibodies (IgG, IgM and IgA) in hamster serum samples was determined using a sandwich-ELISA method (Kit manual; https://pishtazteb.com/wp-content/uploads/2021/04/SARS-Cov-2-Spike-Ab.pdf).

On day 35 post-vaccination, blood samples were collected from the heart of hamsters under intraperitoneal anaesthesia with ketamine-xylazine (K, 150 mg/kg; X, 10 mg/kg). Serum samples were collected in clot activator tubes for the detection of SARS-COV-2 total Antibody. All animal sera were separated and stored at −80° C. until use.

The ELISA kit for antibody detection (SARS-COV-2 RBD Total) was purchased from Pishtazteb Diagnostics Company (Tehran, Iran). In brief, the RBD antigen was coated onto a 96-well plate. 50 μl serum plus 100 μl Enzyme-conjugate were added to each well in duplicate. It was shaken for 30 seconds and incubated at 37° C. for an hour. Next, wells were washed 5 times using washing buffer and 100 μl of chromogen substrate was added to the wells and incubated at room temperature in the dark for 15 min. 100 μl of stop solution was added and the absorbance was read at 450 nm wavelength using an ELISA microplate reader.

The cut-off was measured using the formula Cut-off value being Mean of the negative control group+0.15. The Cut-off Index (COI) is the optical density (OD) of sample/serum Cut-off value. Based on the kit manual, a value less than 0.9 is considered a negative response and a value higher than 1.1 is a positive response.

### 2. SARS-COV-2 Neutralizing Antibody (NA)

The level of SARS-COV-2 NA in hamster serum samples was determined using a competitive method. The competition between the NA and hACE2 with RBD defines the level of NA in serum. The ELISA kit for antibody detection (SARS-COV-2 Neutralizing Antibody) was purchased from Pishtazteb Diagnostics Company (Tehran, Iran). In brief, the RBD immunogen has been coated onto a 96-well plate. 50 μl serum plus 50 μl Conjugate Enzyme were added to each well in duplicate. It was shaken for 15 s and incubated at 37° C. for 30 min. Next, wells were washed 5 times using washing buffer followed by the addition of 100 μl chromogen substrate and incubated at room temperature in the dark for 15 min. 100 μl stop solution was added and the absorbance was read at 450 nm wavelength using an ELISA microplate reader. The concentration of NA was calculated based on the standard curve.

### 3. Live Virus Neutralization Assay (VNA)

Prior to the neutralization assay, sera samples were heat-inactivated at 56° C. for 30 min. Inactivated sera samples were serially diluted 2-fold (range ½- 1/32) in DMEM supplemented with 100 U/mL penicillin, 100 μg/mL streptomycin, and 2 mM glutamine, mixed with SARS-COV-2 isolates and further incubated at 37° C. for 1 h. Each dilution (in duplicate) contains 100 TCID of live viruses. The mixtures were then transferred to Vero E6 cell monolayers (ATCC CRL-1586) and cultured for seven days at 37° C. and 5% CO2. Cytopathic effects of the virus were measured after seven days.

### Statistical Analysis

Graph pad software was applied to statistically analyze body temperature, weight, serum antibody level, NA concentration and the titre of protective antibodies against the virus. All the data presented as mean±SD. The temperature and body weight data were subtracted from the first day and presented as mean±SD. The One-way ANOVA with Tukey post-test was used for statistical analysis. A P value of less than 0.05 was considered statistically significant.

### Results

### 1. Preclinical Evaluation

Two animals in the HMPC60R and HMPC60 groups died and one of them was substituted. Two hamsters that received HPMC60R had severe sores in the i.p injection site of ketamine and Xylazine.

There was no significant difference between the changes of body temperature in all groups on the 2^{nd}, 3^{rd}, 14^{th}, 21^{st} and 35^{th} days (P values=0.4240, 0.7747, 0.4970, 0.8839 and 0.3869, respectively-data not shown).

Statistical analysis also showed there was no significant difference between the changes in body weight on the 2^{nd}, 3^{rd}, 14^{th}, 21^{st} and 35^{th} days (P values=0.3494, 0.7868, 0.7302, 0.2203 and 0.6721, respectively-data not shown).

### 2. SARS-COV-2 Antibody Analysis

The level of total antibody against SARS-COV-2 in hamster serum samples was determined and the cut-off level and index were calculated to determine negative and positive antibody responses.

FIG. 1 shows the outcome of antibody analysis between the groups. There was a significantly higher antibody level in groups HPMC100R compared with HPMC60, HPMC100, HPMC (P<0.05).

While the difference in antibody was not significant when comparing HPMC60 and HPMC100 with control HPMC (P>0.05) and as well as in the comparison of HPMC60 with HPMC100 (P>0.05). Moreover, there was no significant difference in the level of antibodies when comparing HPMC60R with HPMC100R (P>0.05).

Based on the kit manual, the groups with a cut-off index of 1.1 or higher are considered positive. Therefore, all groups except HMPC100R were considered negative at this time point. A point to note is that the cut-off index for group six, that is to say, the hamster subcutaneously injected with inactivated SARS-COV-2 virus, was less than that of intranasally applied HPMC100R and HPMC60R.

### 3. SARS-COV-2 Neutralizing Antibody (NA)

NA analysis was performed to evaluate the protection of the produced antibody in hamsters in competition to hACE2 receptors in humans. The calculated equation for this analysis was “y=−0.346 ln(x)+1.2135” where is y OD of NA in serum and x is concentration of NA in serum. NA evaluation on the 11^{th} and 35^{th} days showed that the P value was 0.4776 and 0.3005, respectively. There was no significant difference between the concentration of NA when serums of hamsters were checked by NA kit.

### 4. Viral Neutralization Analysis (VNA)

VNT analysis was performed to evaluate the protection of the produced antibody in hamsters against live viruses cultured in Vero cells. Results showed TCID50 titer of HPMC100R was significantly higher than all groups including HPMC100, HPMC, HPMC60, HPMC60R, (P<0.001). Moreover, the TCID50 titer of HPMC60R was significantly higher than the HPMC60 (p<0.01), and HPMC (P<0.001).

There was no significant difference between HPMC60R and HPMC100 (P>0.05). Moreover, there was no significant difference between the TCID50 titre of the HPMC and HPMC60 (P>0.05).

### Conclusion

This study aimed to evaluate the efficacy of the intranasal vaccine of the invention for COVID-19. Based on the data, the formulation with the RBD Spike at the concentration of 100 μg (HPMC100R) induced the highest protective antibody titre against the live SARS-COV-2 virus using the VNT test.

Preclinical findings showed that there was no significant difference between the temperature changes and body weight loss in groups (data not shown). Cut-Off index of total antibodies in serum showed that although HPMC100R induced significantly higher total antibody than others, it did not significantly induce higher total antibody than HPMC60R. Based on the kit manual, a cut-off index higher than 1.1 is valuable. Therefore, just the cut-off index of HPMC100R was higher than 1.1 while HPMC60R had a larger SD. NA analysed by NA kit showed that there was no significant difference between the concentrations of neutralizing antibodies in all groups when they were checked by NA kit. Moreover, the results by the NA in the face to the virus in VERO cells indicated in HPMC100R induced a higher level of neutralizing antibodies in hamsters than others. TCID50 and the concentration of neutralizing antibodies in hamsters treated by nasal formulation were significantly higher than the subcutaneously injected killed virus.

In summary, the formulation containing adjuvant-induced higher total antibody and NA than formulation without adjuvant. Moreover, based on studies on rats and hamsters by our group, a higher dose of adjuvant (15 and 30 μg) is recommended. A higher titre of NA using NA kit (NA=25 μg/ml) and live SARS-COV-2 virus (TCID50-44) were seen in rats treated with HPMC50R.

### APPENDIX 1

The TCID50 (Median Tissue Culture Infectious Dose) assay is one method used to verify the viral titre of a testing virus.

Host tissue cells are cultured on a well plate titre, and then varying dilutions of the testing viral fluid are added to the wells.

After incubation, the percentage of infected wells is observed for each dilution, and the results are used to calculate the TCID50 value.

### Rabbit Model

### Introduction

Following on from the research on hamsters and rats provided hereinabove, research on rabbit was aimed to assess HPMC powder with spike RBD protein plus Influenza A (H1N1) antigen and adjuvant as prevention of COVID-19 infection. In the present study, the adjuvant MF59 was added to the formulation beside VaccigradeR848™ (R848) adjuvant to enhance the antibody response in rabbits. Moreover, Influenza A (H1N1) was added creating a multivalent vaccine.

In the first phase rat study reported herein, it was concluded that the formulations with the RBD Spike at the concentration of 100 μg+7.5 μg R848 induced higher titer of protective antibodies against the live virus. Therefore, to enhance the immunogenicity and protective efficacy of the formulation, an adjuvant cocktail at higher concentration and follow-up, up to day 35 was performed in rats. Results indicated in the proposed formulations with the RBD Spike at the concentration of 50 μg induced higher titer of protective antibodies against the live virus than 20 μg. To sum things up, 50 μg spike plus adjuvant-induced valuable protective antibodies (1/44) against live virus. Deletion of adjuvant or low concentration of adjuvant in the formulations even at higher concentrations of spike protein and longer time-points do not induce valuable protective antibodies. To enhance the immunogenicity and protective efficacy of the formulation, an adjuvant cocktail at a higher concentration of 30 μg and follow-up up to 35 days, was suggested. In the second phase, the formulations with 30 μg Adjuvant and follow-up up to 35 days were studied. In the present stage on rabbits, to enhance the immune response against the SARS-COV-2 virus and neutralizing antibodies, a further adjuvant, MF59, was added to the formulation.

Hypothesis: The cocktail of RBD antigen (60 μg), both adjuvants (R848 and MF59), Influenza A (H1N1) and HPMC powder will promote passive immunity in rabbits. Will it induce neutralizing antibody which has a protective potential against reinfection by the SARS-COV-2 virus?

### 1. Materials and Methods

### 1.1 Vaccine Formulations

The commercialized vaccines are in liquid form therefore, we had to prepare the formulations in liquid form. All were admixed together immediately prior to nasal delivery as a liquid. Three groups were defined to evaluate total antibodies, neutralizing antibodies, and antibody protection against SARS-COV-2 in rabbit animal models as follows:
G1. 60 μg RBD+500 μg HPMC+30 μg adjuvant (VaccigradeR848)+0.25 ml MF590+7.5 μg Influenza A (H1N1) antigen (in just one nostril). The Powder form (RP*) *RP: Liquid MF590 and Influenza antigen were in liquid form applied and then after 30 s, all except adjuvants and influenza antigen mixed together and applied in powder form.
G2. 60 μg RBD+500 μg HPMC+30 μg adjuvant (VaccigradeR848)+0.25 ml MF59+7.5 μg Influenza A (H1N1) antigen (in just one nostril). The liquid form (RL*). *RL: All materials admixed together and made up to 0.25 ml. mixture applied as a liquid.
G3. 500 μg HPMC powder.

### 1.2 Ethics Statement

The animal experiments were approved by the ethical committee of the Iran University of Medical Sciences (IR.IUMS.REC.1401.460). Experiments with SARS-COV-2 (Virus neutralization assay (VNA)) were performed at the Biosafety Level-3 (BSL-3) facility.

### 1.3 Rabbit Animal Model

Rabbits were used for the evaluation of the newly formulated commercialized vaccines. All animals (3-month-old) were obtained from the animal house. The animals were maintained at a controlled temperature of 24±1° C. with a 12-12 h light-dark cycle (light cycle, 07:00-19:00), and were allowed free access to water and standard chow and libitum. Rabbits were randomly divided into three groups (n=3/group). Rabbits intranasally received vaccine formulations three times at intervals of fourteen days: 0, 14 and 28^{th} days. Animals were kept until the 42nd-day post-treatment.

### 1.4 Physiological Evaluation

The animals were followed up for 42 days. Mortality was recorded. Body temperature and weight were monitored at 1, 2, 3, 14, 15, 16, 28, 29, 30 and 42 days in all groups. The data was subtracted from the first day and mean±SE was reported.

### 1.5 SARS-COV-2 Antibody Kit Development and Analysis

The level of anti-SARS-COV-2 antibodies in rabbit serum samples was determined using a sandwich-ELISA method. On days 28 and 42 post-vaccination, blood samples were collected from the ears of rabbits under intraperitoneal sedation with ketamine-xylazine (K, 50 mg/kg; X, 5 mg/kg). Serum samples were collected in clot activator tubes for the detection of SARS-COV-2 total Antibody. All animal sera were separated and stored at −80° C. until used.

The Eliza kit for antibody detection (SARS-COV-2 RBD Total) was purchased. In brief, RBD antigen has been coated in 96 well-plates. 50 μl serum plus 100 μl Enzyme-conjugate were added to each well in duplicate. It was shaken for the 30 s and incubated at 37° C. for 1 h. Then, wells were washed 5 times using washing buffer and 100 μl chromogen substrate was added to wells and incubated at room temperature in dark for 15 min.

100 μl Stop solution was added and the absorbance was read at 450 nm wavelength using an Elisa microplate reader.

The mean±SE was reported, and the cut-off was measured using the formula. Cut-off value is Mean of the negative control group+0.15. The Cut-off Index (COI) is the OD of the sample/Cut-off value. A value less than 0.9 is a negative response and a higher than 1.1 is a positive response.

### 1.6 SARS-COV-2 Neutralizing Antibody

The level of SARS-COV-2 Neutralizing antibodies in rabbit serum samples was determined using a competitive method. In fact, the competition between the neutralizing antibody and ACE2 with RBD defines the level of neutralizing antibody in serum. The Eliza kit for antibody detection (SARS-COV-2 Neutralizing Ab) was used. In brief, RBD antigen has been coated in 96 well-plates. 50 μl serum plus 50 μl Conjugate Enzyme were added to each well in duplicate. It was shaken for 15 s and incubated at 37° C. for 30 min. Then, wells were washed 5 times using washing buffer and, 100 μl chromogen substrate was added to wells and incubated at room temperature in dark for 15 min. 100 μl Stop solution was added and the absorbance was read at 450 nm wavelength using an Elisa microplate reader. The concentration of neutralizing antibodies was calculated based on the standard curve.

### 1.7 Hemagglutination Inhibition (HI)

Rabbits will be evaluated for Influenza antibodies on 28 and 42 days using hemagglutination inhibition (HIA) assay.

### 2.9 Statistical Analysis

Graph pad software was applied to statistically analyze body temperature, weight, serum antibody level, neutralizing antibody concentration and the titer of protective antibodies against the virus. Experiments were performed as mean±SE. The One-way ANOVA with Tukey post-test was used for statistical analysis. A P value of less than 0.05 was considered statistically significant.

### Results

### 3.1 Physiological Evaluation

Body temperatures were monitored on 1, 2, 3, 14, 15, 16, 28, 29, 30 and 42^{nd} days, there was no statistically significant difference between the body temperature in groups or over the period of study (P>0.05).

Body weight loss was monitored in rabbits on the 2, 3, 14, 15, 16, 28, 29, 30 and 42nd days. Statistical analysis on the 2, 3, 14, 15, 16, 28, 29, 30 and 42^{nd} days indicated that the P>0.05; was considered not a statistically significant difference in changes in the weight over 42 days or between groups.

### 3.2 SARS-COV-2 Antibody Analysis

The level of total antibody against SARS-COV-2 in rabbit serum samples was determined. The induced antibody response on the 28^{th} day was low (FIG. 6), while R-Powder and Liquid formulations induced IgA, IgG and IgM antibodies in rabbits on the 42^{nd} day were significantly higher than the control group. However, R-Powder induced significantly higher total antibody than liquid formulation.

### 3.3. SARS-COV-2 Neutralizing Antibody.

NA analysis was performed to evaluate the protection of the produced antibody in rabbits in competition with hACE2 receptors in humans. The calculated equation for this analysis on the 28^{th} day was “Y=−0.313 ln(X)+1.0962” where is Y optical density (OD) and X is concentration (standard curve FIG. 7A). Neutralizing antibody evaluation on the 28^{th} day (FIG. 8A) showed that the P value was less than 0.05 and was a significant difference between the concentration of neutralizing antibodies in R-powder compared to the other groups when they were checked by the NA kit.

The calculated equation for this analysis on the 42^{nd} day was “Y=−0.282 ln(X)+1.0878” (standard curve FIG. 7B). Neutralizing antibody evaluation on the 42^{nd} day (FIG. 8B) showed that R-Powder and R-Liquid induced significant neutralizing antibodies compared to the control group while the concentration of neutralizing antibodies was significantly higher in the R-Powder compared to the R-Liquid group (P<0.05).

### 3.4 Hemagglutinin Inhibition (HI) Analysis.

HI, analysis was performed on the 28^{th} and 42^{nd} days post-treatment. Results showed that there was a significant difference between the titre of Hemagglutinin antibodies in rabbits vaccinated with R-Powder against the Influenza antigen on the 28^{th} and 42^{nd} days compared to the other groups.

### Conclusion

The aim of this study was to evaluate the efficacy of test COVID-19 vaccines administered via the intranasal route in rabbits. In the present study, an extra adjuvant, MF59, in addition to R848 was added to enhance the antibody response against SARS-COV-2 in rabbits. Moreover, HA of the influenza H1N1 virus was added to the formulation as a multivalent vaccine. Results showed that there were no significant differences between the body temperature and weight among the two vaccines and HPMC as the control group for 42 days. Furthermore, although, none of the formulations induced antibody response against SARS-COV-2 in rabbits on the 28th day, the R-powder test vaccine induced IgM, IgG and IgA antibodies and neutralizing antibodies against SARS-COV-2 virus on the 42^{nd} day. In conclusion, it might be said that powder formulation vaccines could induce protective antibodies against SARS-CoV-2 in rabbits. Moreover, based on raw data derived from HI antibody titre in powder test Vaccine, it may be said that it induces hemagglutinin antibody as well.

### REFERENCES

1. Tavakol S, Tavakol H, Alavijeh M S, Seifalian A. The World Against Versatile SARS-Cov-2 Nanomachines; Mythological or Reality? Current Stem Cell Research & Therapy. 2021.
2. WHO. Tracking SARS-COV-2 variants. 6 Jul. 2021.
3. Tavakol S, Alavijeh M S, Seifalian A M. COVID-19 Vaccines in Clinical Trials and their Mode of Action for Immunity against the Virus. Current pharmaceutical design. 2021; 27 (13): 1553-63.
4. Roper R L, Rehm K E. SARS vaccines: where are we? Expert review of vaccines. 2009; 8 (7): 887-98.
5. Bolles M, Deming D, Long K, Agnihothram S, Whitmore A, Ferris M, et al. A double-inactivated severe acute respiratory syndrome coronavirus vaccine provides incomplete protection in mice and induces increased eosinophilic proinflammatory pulmonary response upon challenge. Journal of virology. 2011; 85 (23): 12201-15.
6. Wang Q, Zhang L, Kuwahara K, Li L, Liu Z, Li T, et al. Immunodominant SARS coronavirus epitopes in humans elicited both enhancing and neutralizing effects on infection in non-human primates. ACS infectious diseases. 2016; 2 (5): 361-76.
7. Tseng C-T, Sbrana E, Iwata-Yoshikawa N, Newman P C, Garron T, Atmar R L, et al. Immunization with SARS coronavirus vaccines leads to pulmonary immunopathology on challenge with the SARS virus. PloS one. 2012; 7 (4).
8. Sepkowitz K. When can a Covid-19 vaccine really be ready? CNN. 2020.
9. Peeples L. News Feature: Avoiding pitfalls in the pursuit of a COVID-19 vaccine. Proceedings of the National Academy of Sciences. 2020; 117 (15): 8218-21.
10. Wan Y, Shang J, Sun S, Tai W, Chen J, Geng Q, et al. Molecular mechanism for antibody-dependent enhancement of coronavirus entry. Journal of virology. 2020; 94 (5): e02015-19.
11. B.1.1.7: What We Know About the Novel SARS-COV-2 Variant [Internet]. American Society for Microbiology. 2021. Available from: https://asm.org/Articles/2021/January/B-1-1-7-What-We-Know-About-the-Novel-SARS-COV-2-Va.
12. Andrew Rambaut, Nick Loman, Oliver Pybus, Wendy Barclay, Jeff Barrett, Alesandro Carabelli, et al. Preliminary genomic characterisation of an emergent SARS-COV-2 lineage in the UK defined by a novel set of spike mutations. wwwvirologicalorg. 2020.
13. Fratev F. The SARS-COV-2 S1 spike protein mutation N501Y alters the protein interactions with both hACE2 and human derived antibody: A Free energy of perturbation study. bioRxiv. 2020.
14. Fratev F. The N501Y and K417N mutations in the spike protein of SARS-COV-2 alter the interactions with both hACE2 and human derived antibody: A Free energy of perturbation study. bioRxiv. 2020.
15. Song Y, Zhou Y, Chen L. Cellulose-based polyelectrolyte complex nanoparticles for DNA vaccine delivery. Biomaterials science. 2014; 2 (10): 1440-9.
16. Arthanari S, Mani G, Peng M M, Jang H T. Chitosan-HPMC-blended microspheres as a vaccine carrier for the delivery of tetanus toxoid. Artificial cells, nanomedicine, and biotechnology. 2016; 44 (2): 517-23.
17. Jeon W, Kim Y-C, Hong M, Rejinold S, Park K, Yoon I, et al. Microcrystalline cellulose for delivery of recombinant protein-based antigen against erysipelas in mice. BioMed research international. 2018; 2018.

1-48. (canceled)

49. A respiratory virus vaccine composition for nasal administration to a mammal comprising a hygroscopic gel-forming material, at least one isolated bioactive respiratory virus immunogen, and an adjuvant.

50. A respiratory virus vaccine composition according to claim 49, wherein the hygroscopic gel-forming material is selected from carrageenan and hydroxypropylmethyl cellulose.

51. A respiratory virus vaccine composition according to claim 49, wherein the said composition is in the form of a dry particulate powder, the said composition being in the form of particles having a mean particle size diameter of ≥10 μm to ≤400 μm, wherein the said immunogen is at least one viral protein in lyophilised form.

52. A respiratory virus vaccine composition according to claim 51 in the form of a dry particulate powder comprising or consisting of:
i) dry powder hygroscopic gel-forming material particles; and
ii) at least one isolated bioactive respiratory virus immunogen; and
iii) an adjuvant,
wherein the said respiratory virus vaccine composition has a mean viscosity within the range 22 Pa·s+/−2 Pa·S to 40 Pa·s.+/−5 Pa·S at 20° C. in a 3.6% aqueous solution.

53. A composition according to claim 51, wherein the at least one bioactive respiratory virus immunogen is selected from isolated immunogenic viral proteins of viruses selected from the group: coronaviruses, influenza viruses such as strains of influenza A, parainfluenza viruses, metapneumoviruses, respiratory syncytial viruses, rhinoviruses and bocaviruses.

54. A composition according to claim 53, wherein the said at least one isolated bioactive respiratory virus immunogen is selected from isolated immunogenic viral proteins of a coronavirus species selected from SARS-COV, MERS-COV, SARS-COV-2, HCov-NL63, HCov-OC43, CoV-HKU1, HCov-229E and mutant strains thereof; and from immunogenic viral proteins of an influenza A virus species selected from H1N1, H5N1, and H3N2 and mutant strains thereof.

55. A composition according to claim 53, wherein the said at least one isolated bioactive respiratory virus immunogen is selected from the MERS spike protein (MERS-S) and immunogenic parts thereof, SARS-COV-2 spike protein and immunogenic parts thereof, the trimeric spike protein and immunogenic parts thereof, the SARS-COV-2 nucleocapsid protein and immunogenic parts thereof, spike protein receptor binding domain (S-RBD) and immunogenic parts thereof, the S1 subunit of the spike(S) protein and immunogenic parts thereof such as the C-domain, and the transmembrane protease TMPRSS2 and immunogenic components thereof; the influenza virus A computationally optimized broadly reactive antigen (COBRA) neuraminidase (NA) surface protein, (i.e. the N1-I COBRA NA antigen), haemagglutinin antigens (HA) for the variable head domain of HA, the HA stalk structure, composed of portions of HA1 and all of HA2, headless HA, chimeric HA, mosaic HA, computationally-optimized broadly reactive antigens (COBRA), and “breathing” HA; the respiratory syncytial virus (RSV) G and F glycoproteins proteins from RSV A and RSV B such as PFP-1 and/or PFP-2 and/or PFP-3 a, co-purified F, G and matrix (M) proteins, and BBG2Na, a peptide from the G glycoprotein central conserved region of the G glycoprotein, a prokaryotically expressed fusion protein that consists of the central conserved region of the G glycoprotein from the RSV A Long strain (residues 130-230) fused to the albumin-binding domain of streptococcal protein G; the parainfluenza virus (PIV) proteins F and N proteins of human PIV-3; the human metapneumovirus (HMPV) proteins such as protein F; rhinovirus (RV) proteins such as VP1, and VP3 from RV14 and conserved regions of VP4, RV polyprotein encompassing VP4 and VP2 (known as VP0), and RV16 VP0-VP0, and the Nlm-II region of VP2; and human bocavirus (HBoV) recombinant protein HBoV viral capsid protein 2-virus like particles.

56. A composition according to claim 53, wherein the adjuvant is selected from one or more of R848 VACCIGRADE™, alum salts, such as potassium aluminium sulphate, aluminium hydroxide, aluminium phosphate, ASO4 (3-O-desacyl-4′-monophosphoryl lipid A [MPL] adsorbed on alum), ASO3 (squalene oil-in-water emulsion containing α-tocopherol [vitamin E], ASO1 (TLR4 ligand and a purified saponin fraction [QS-21, a triterpene glycoside] are formulated together in liposomes in the presence of cholesterol), MF59 (oil-in-water emulsion formed squalene oil stabilised in aqueous buffer by non-ionic surfactants Tween 80 and Span 85), cytosine phosphoguanosine 1018 (CpG 1018: a TLR9 22-mer unmethylated CpG-B class single stranded oligonucleotide), MATRIX-M™ (Novavax proprietary adjuvant MATRIX-M™, a saponin-based adjuvant consisting of two populations of individually formed 40 nm sized Matrix particles, each with a different and well characterized saponin fraction with complementary properties (Fraction-A and Fraction-C, respectively, suitable proportions in Matrix-M are 85% Matrix-A and 15% Matrix-C), the Matrix particles are formed by formulating purified saponin from Q. saponaria Molina with cholesterol and phospholipid; and Toll-like receptor 7/Toll-like receptor 8 (TLR7/TLR8) ligand adsorbed in alum.

57. A composition according to claim 56, wherein the adjuvant is selected from an alum salt, R848 VACCIGRADE™, MF59, CpG 1018, and MATRIX-M™.

58. A composition according to claim 57, wherein the adjuvant is selected from an alum salt powder, MF 59, and R848 VACCIGRADE™ in lyophilised form.

59. A composition according to claim 49, wherein the mammal is selected from a mouse, a hamster, a rat, a ferret, an ape, a monkey, a horse, a camel, and a human being.

60. A respiratory virus vaccine composition for nasal administration to a mammal according to claim 49, wherein the said composition is in the form of a gel comprising or consisting of a hygroscopic gel-forming material, at least one isolated bioactive respiratory virus immunogen, as defined in claim 54, and an adjuvant as defined in claim 56, and physiological saline.

61. A respiratory virus vaccine composition for nasal administration to a mammal comprising:
a first formulation comprising hygroscopic gel-forming material particles and at least one bioactive respiratory virus immunogen as defined in claim 54 or an immunological fragment thereof; and
a second formulation comprising an adjuvant as defined in claim 56, wherein the first formulation and second formulation are administered together or sequentially to a patient via the nasal route.

62. A respiratory virus vaccine composition according to claim 61, wherein the hygroscopic gel-forming material is selected from carrageenan and hydroxypropylmethyl cellulose.

63. A method of making the respiratory virus vaccine composition as defined in claim 49 comprising:
1. adding lyophilised immunogen as defined in claim 54 to hydroxypropyl methylcellulose powder at a temperature≥10° to ≤18° C.;
2. gently blending the two ingredients of 1) in a blending machine; and
3. adding lyophilised adjuvant as defined in claim 56 and further blending.

64. A method of making the respiratory virus vaccine composition as defined in claim 60 comprising:
1. adding lyophilised immunogen as defined in claim 54 to hydroxypropyl methylcellulose powder at a temperature≥10° to ≤18° C.;
2. gently blending the two ingredients of 1) in a blending machine; and
3. adding an adjuvant as defined in claim 56 and further blending;
4. adding physiological saline to the blended product of step 3).

65. A respiratory virus vaccine composition according to claim 49 in a method of boosting or further potentiating the treatment of an immunological response in a mammal that has had at least a first prior vaccination event against a respiratory virus disease.

66. A respiratory virus vaccine composition according to claim 65, wherein the respiratory virus disease is selected from Covid-19 and influenza.

67. A respiratory virus vaccine composition according to claim 66, wherein the respiratory virus disease is influenza.

68. A respiratory virus vaccine composition in the form of a dry particulate powder comprising or consisting of:
i) dry powder hydroxypropyl methylcellulose (HPMC) particles; and
ii) at least one isolated bioactive respiratory virus immunogen; and
iii) an adjuvant,
wherein the mean viscosity of the dry powder HPMC particles per se lies within 10 to 20 Pa·S, at 20° C. in a 2% aqueous solution, such as, 10 Pa·S to 17 Pa·S at 20° C. in a 2% aqueous solution.
